(19) United States
(12) Patent Application Publication
(10) Pub. No.: US 2025/0057478 A1
(43) Pub. Date: Feb. 20, 2025

(54) FORCE APPLICATORS FOR UPPER TORSO TISSUE IN WEARABLE CARDIAC DEVICES

(71) Applicant: ZOLL Medical Corporation, Chelmsford, MA (US)

(72) Inventors: Saundra L. Penkunas, Portland, OR (US); Philip C. Skalos, Pittsburgh, PA (US); Christopher L. Swenglish, Connellsville, PA (US); Krista M. Bacchioni, New Albany, OH (US); Stephanie K. Muhlenfeld, Portland, OR (US)

(21) Appl. No.: 18/800,718

(22) Filed: Aug. 12, 2024

Related U.S. Application Data
(60) Provisional application No. 63/519,383, filed on Aug. 14, 2023.

(51) Int. Cl. A61B 5/00; A41C 3/08; A61B 5/256; A61B 5/282; A61B 5/332

(52) U.S. Cl. CPC A61B 5/6823; A41C 3/08; A61B 5/256; A61B 5/282; A61B 5/332; A61B 5/4836; A61B 5/6831; A61B 5/6843

(57) ABSTRACT

An ambulatory patient monitoring and treatment device configured to be worn continuously is provided. The device includes sensing electrodes, therapy electrodes, and cardioprotective apparel for wear about the torso. The cardioprotective apparel includes a cardioactive band configured to support the sensing electrodes and anterior therapy electrode(s) such that, when worn, the sensing electrodes are disposed on predetermined sensing anatomical regions and the anterior therapy electrode(s) are disposed on an anterior therapy electrode placement region. The cardioprotective apparel also includes at least two shoulder harnesses. Each of the at least two shoulder harnesses includes a force applicator having an elasticity configured to shift breast tissue away from at least one sensing anatomical region and/or the anterior therapy electrode placement region, thereby facilitating the anterior therapy electrode(s) and the sensing electrodes in lying flush with a patient's skin. The device also includes a controller and associated circuitry operably connected to the electrodes.

### CROSS-REFERENCE TO RELATED APPLICATIONS

This application claims priority to U.S. Provisional Patent Application Ser. No. 63/519,383, filed on Aug. 14, 2023, titled “FORCE APPLICATORS FOR UPPER TORSO TISSUE IN WEARABLE CARDIAC DEVICES,” which is hereby incorporated by reference in its entirety.

### BACKGROUND

The present disclosure relates to a wearable cardiac treatment system configured to treat cardiac arrhythmias occurring in ambulatory patients.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and/or asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients may be prescribed to wear cardiac treatment devices for extended periods of time. Cardiac treatment devices may provide defibrillation shocks to the patient if an abnormal cardiac rhythm is detected. The abnormal cardiac rhythm is detected using electrocardiogram (ECG) electrodes, and the defibrillation shocks are provided using therapy electrodes.

### SUMMARY

In one or more examples, an ambulatory patient monitoring and treatment device configured to be worn continuously by a patient is provided. The device includes a plurality of ECG sensing electrodes configured to sense cardiac activity of the patient, a plurality of therapy electrodes configured to deliver one or more electrical therapeutic shocks to the patient, and cardioprotective apparel configured to be donned about an upper torso region of the patient. The cardioprotective apparel includes a cardioactive band configured to be disposed on an inferior end of the upper torso region of the patient and support at least one anterior therapy electrode of the plurality of therapy electrodes such that the at least one anterior therapy electrode is disposed on an anterior therapy electrode placement region when the device is worn by the patient, and the plurality of ECG sensing electrodes distributed in a spaced apart configuration such that the plurality of ECG sensing electrodes are disposed on predetermined ECG sensing anatomical regions when the device is worn by the patient. The cardioprotective apparel also includes at least two shoulder harnesses attached to the cardioactive band and configured to support the cardioprotective apparel on the patient's shoulders. Each of the at least two shoulder harnesses includes a force applicator having an elasticity configured to shift breast tissue of the upper torso region of the patient away from at least one of the anterior therapy electrode placement region on the patient or at least one of the predetermined ECG sensing anatomical regions on the patient. The shifting of the breast tissue of the upper torso region of the patient is configured to facilitate the at least one anterior therapy electrode and the plurality of ECG sensing electrodes to lie in a flush manner with the patient's skin when the device is worn by the patient. The device also includes a device controller and associated circuitry operably connected to the plurality of ECG sensing electrodes and the plurality of therapy electrodes and configured to determine whether the patient is experiencing a treatable arrhythmia based on the sensed cardiac activity and instruct the delivery of the one or more electrical therapeutic shocks to the patient via the plurality of therapy electrodes in response to determining that the patient is experiencing a treatable arrhythmia.

Implementations of the ambulatory patient monitoring and treatment device configured to be worn continuously by the patient can include one or more of the following features. Each force applicator includes a sling portion configured to shift the breast tissue of the upper torso region of the patient. The sling portion includes a fabric having the elasticity configured to shift the breast tissue of the upper torso region of the patient. The fabric of the sling portion include at least one of a mesh, a knit, or a stretch woven. The sling portion includes a plurality of layers bonded together. The at least two shoulder harnesses are configured to be disposed along lateral ends of the upper torso region of the patient when the device is worn by the patient such that each sling portion is configured to contact a lateral portion of the patient's breast tissue. Each force applicator includes at least one of a cut, a pattern, a construction, a material, or a material elasticity of the corresponding shoulder harness. Each force applicator is configured to provide less than 0.65 psi of pressure to the breast tissue of the upper torso region of the patient.

The cardioprotective apparel is configured in a size selected from a range of sizes. Each force applicator is configured to shift the breast tissue of the upper torso region of the patient to displace the breast tissue by an amount according to the size of the cardioprotective apparel. The at least two shoulder harnesses are configured to minimize interference with a bra worn by the patient concurrently with the monitoring and treatment device. The at least two shoulder harnesses are configured to be disposed along lateral ends of the upper torso region of the patient when the device is worn by the patient to minimize overlap between the bra worn by the patient and the at least two shoulder harnesses. The at least two shoulder harnesses are configured to stabilize the breast tissue within the bra when the bra is worn over the monitoring and treatment device.

The cardioprotective apparel includes a plurality of zones. Each zone includes a predetermined elasticity for the respective zone. The plurality of zones includes a zone corresponding to a portion of patient anatomy. The predetermined elasticity of the zone corresponding to the portion of patient anatomy is configured to accommodate the corresponding portion of patient anatomy. The plurality of zones includes a zone corresponding to one or more of at least one ECG sensing electrode or at least one therapy electrode. The predetermined elasticity of the zone corresponding to the one or more of the at least one sensing electrode or the at least one therapy electrode is configured to provide a predetermined compression to the one or more of the at least one sensing electrode or the at least one therapy electrode. The elasticity of each zone is at least partially controlled by a knit of the cardioprotective apparel in the zone. The elasticity of each zone is at least partially controlled by a material of the cardioprotective apparel in the zone. The cardioactive band includes one zone. The cardioactive band includes two or more zones. The plurality of zones includes a first zone including each of the force applicators configured to shift the breast tissue of the upper torso region of the patient.

The cardioactive band includes a clasp shaped to accommodate anterior torso patient anatomy. The cardioactive band includes fabric forming at least a portion of the cardioactive band. A bottom edge of the clasp is offset from a bottom edge of the fabric forming the at least portion of the cardioactive band. The clasp is configured in a conical closure configuration.

A length of an anterior portion of the cardioactive band is between about 8.5 inches and about 19 inches. A length of a posterior portion of the cardioactive band is between about 17 inches and about 38 inches. The cardioactive band further includes at least one anterior therapy electrode receptacle configured to receive a respective anterior therapy electrode and resist a transverse rotational force exerted by underlying tissue of the upper torso region of the patient such that the respective anterior therapy electrode is configured to lie in the flush manner with the patient's skin when the device is worn by the patient. The at least one anterior therapy electrode receptacle includes at least one anterior pocket disposed on an anterior portion of the cardioactive band. The cardioprotective apparel includes an inner side configured to face the patient's skin and an outer side configured to face away from the patient's skin. The at least one anterior pocket disposed on the anterior portion of the cardioactive band is configured to be accessible from the outer side of the cardioprotective apparel. The cardioprotective apparel includes an inner side configured to face the patient's skin and an outer side configured to face away from the patient's skin. The at least one anterior pocket disposed on the anterior portion of the cardioactive band is configured to be accessible from the inner side of the cardioprotective apparel. Each anterior pocket is pre-gathered, the pre-gathering facilitating the respective anterior therapy electrode in lying in the flush manner with the patient's skin when the respective anterior therapy electrode is received into the anterior pocket and the device is worn by the patient.

Each of the at least two shoulder harnesses includes a strap configured to adjust at least one of a length or a shape of the respective shoulder harness. Each strap is configured to run between layers of fabric of the cardioprotective apparel. The straps are configured to crisscross along a posterior portion of the cardioprotective apparel. Each strap includes at least a portion of an adjustment mechanism configured to adjust the at least one of the length or shape of the respective shoulder harness. Each adjustment mechanism is configured to adjust a shape of the force applicator configured to shift the breast tissue of the upper torso region of the patient. An additional portion of each adjustment mechanism is located on the cardioactive band.

The cardioactive band is configured to be contoured to the upper torso region of the patient. The cardioactive band is configured to be contoured to accommodate a front torso projection. The cardioactive band is configured to be contoured to accommodate a female front torso projection. The cardioactive band is configured to be contoured to accommodate a male front torso projection.

In one or more examples, an ambulatory patient monitoring and treatment device configured to be worn continuously by a patient is provided. The device includes a plurality of ECG sensing electrodes configured to sense cardiac activity of the patient, a plurality of therapy electrodes configured to deliver one or more electrical therapeutic shocks to the patient, and cardioprotective apparel configured to be donned about an upper torso region of the patient. The cardioprotective apparel includes a cardioactive band configured to be disposed on an inferior end of the upper torso region of the patient and support at least one anterior therapy electrode of the plurality of therapy electrodes such that the at least one anterior therapy electrode is disposed on an anterior therapy electrode placement region when the device is worn by the patient, and the plurality of ECG sensing electrodes distributed in a spaced apart configuration such that the plurality of ECG sensing electrodes are disposed on predetermined ECG sensing anatomical regions when the device is worn by the patient. The cardioprotective apparel also includes at least two shoulder harnesses attached to the cardioactive band and configured to support the cardioprotective apparel on the patient's shoulders. Each of the at least two shoulder harnesses is configured to shift breast tissue of the upper torso region of the patient away from at least one of the anterior therapy electrode placement region on the patient or at least one of the predetermined ECG sensing anatomical regions on the patient. The shifting of the breast tissue of the upper torso region of the patient is configured to facilitate the at least one anterior therapy electrode and the plurality of ECG sensing electrodes to lie in a flush manner with the patient's skin when the device is worn by the patient. The device also includes a device controller and associated circuitry operably connected to the plurality of ECG sensing electrodes and the plurality of therapy electrodes and configured to determine whether the patient is experiencing a treatable arrhythmia based on the sensed cardiac activity and instruct the delivery of the one or more electrical therapeutic shocks to the patient via the plurality of therapy electrodes in response to determining that the patient is experiencing a treatable arrhythmia.

Implementations of the ambulatory patient monitoring and treatment device configured to be worn continuously by the patient can include one or more of the following features. Each of the at least two shoulder harnesses includes a sling portion configured to shift the breast tissue of the upper torso region of the patient. The sling portion includes a fabric having an elasticity configured to shift the breast tissue of the upper torso region of the patient. The fabric of the sling portion includes at least one of a mesh, a knit, or a stretch woven. The sling portion includes a plurality of layers bonded together. The at least two shoulder harnesses are configured to be disposed along lateral ends of the upper torso region of the patient when the device is worn by the patient such that each sling portion is configured to contact a lateral portion of the patient's breast tissue.

Each of the at least two shoulder harnesses includes a force applicator configured to shift the breast tissue of the upper torso region of the patient. Each force applicator is configured as a sling portion configured to shift the breast tissue of the upper torso region of the patient. Each force applicator includes at least one of a cut, a pattern, a construction, a material, or an elasticity of the corresponding shoulder harness. Each force applicator is configured to provide less than 0.65 psi of pressure to the breast tissue of the upper torso region of the patient.

The cardioprotective apparel is configured in a size selected from a range of sizes. The at least two shoulder harnesses are configured to shift the breast tissue of the upper torso region of the patient to displace the breast tissue by an amount according to the size of the cardioprotective apparel. The at least two shoulder harnesses are configured to minimize interference with a bra worn by the patient concurrently with the monitoring and treatment device. The at least two shoulder harnesses are configured to be disposed along lateral ends of the upper torso region of the patient when the device is worn by the patient to minimize overlap between the bra worn by the patient and the at least two shoulder harnesses. The at least two shoulder harnesses are configured to stabilize the breast tissue within the bra when the bra is worn over the monitoring and treatment device.

The cardioprotective apparel includes a plurality of zones. Each zone includes a predetermined elasticity for the respective zone. The plurality of zones includes a zone corresponding to a portion of patient anatomy. The predetermined elasticity of the zone corresponding to the portion of patient anatomy is configured to accommodate the corresponding portion of patient anatomy. The plurality of zones includes a zone corresponding to one or more of at least one ECG sensing electrode or at least one therapy electrode. The predetermined elasticity of the zone corresponding to the one or more of the at least one sensing electrode or the at least one therapy electrode is configured to provide a predetermined compression to the one or more of the at least one sensing electrode or the at least one therapy electrode. The predetermined elasticity of each zone is at least partially controlled by a knit of the cardioprotective apparel in the zone. The predetermined elasticity of each zone is at least partially controlled by a material of the cardioprotective apparel in the zone. The cardioactive band includes one zone. The cardioactive band includes two or more zones. The plurality of zones includes a first zone including at least a portion of each of the at least two shoulder harnesses that is configured to shift the breast tissue of the upper torso region of the patient.

The cardioactive band includes a clasp shaped to accommodate anterior torso patient anatomy. The cardioactive band includes fabric forming at least a portion of the cardioactive band. A bottom edge of the clasp is offset from a bottom edge of the fabric forming the at least portion of the cardioactive band. The clasp is configured in a conical closure configuration.

A length of an anterior portion of the cardioactive band is between about 8.5 inches to and about 19 inches. A length of a posterior portion of the cardioactive band is between about 17 inches and about 38 inches. The cardioactive band further includes at least one anterior therapy electrode receptacle configured to receive a respective anterior therapy electrode and resist a transverse rotational force exerted by underlying tissue of the upper torso region of the patient such that the respective anterior therapy electrode is configured to lie in the flush manner with the patient's skin when the device is worn by the patient. The at least one anterior therapy electrode receptacle includes at least one anterior pocket disposed on an anterior portion of the cardioactive band. The cardioprotective apparel includes an inner side configured to face the patient's skin and an outer side configured to face away from the patient's skin. The at least one anterior pocket disposed on the anterior portion of the cardioactive band is configured to be accessible from the outer side of the cardioprotective apparel. The cardioprotective apparel includes an inner side configured to face the patient's skin and an outer side configured to face away from the patient's skin. The at least one anterior pocket disposed on the anterior portion of the cardioactive band is configured to be accessible from the inner side of the cardioprotective apparel. Each anterior pocket is pre-gathered, the pre-gathering facilitating the respective anterior therapy electrode in lying in the flush manner with the patient's skin when the respective anterior therapy electrode is received into the anterior pocket and the device is worn by the patient.

Each of the at least two shoulder harnesses includes a strap configured to adjust at least one of a length or a shape of the respective shoulder harness. Each strap is configured to run between layers of fabric of the cardioprotective apparel. The straps are configured to crisscross along a posterior portion of the cardioprotective apparel. Each strap includes at least a portion of an adjustment mechanism configured to adjust the at least one of the length or shape of the respective shoulder harness. Each adjustment mechanism is configured to adjust a shape of a portion of the respective shoulder harness configured to shift the breast tissue of the upper torso region of the patient. An additional portion of each adjustment mechanism is located on the cardioactive band.

The cardioactive band is configured to be contoured to the upper torso region of the patient. The cardioactive band is configured to be contoured to accommodate a front torso projection. The cardioactive band is configured to be contoured to accommodate a female front torso projection. The cardioactive band is configured to be contoured to accommodate a male front torso projection.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example ambulatory patient monitoring and treatment device.

FIG. 2 depicts an example of cardioprotective apparel for a monitoring and treatment device.

FIG. 3 depicts an example electrode belt for a monitoring and treatment device.

FIG. 4 depicts an example device monitor for a monitoring and treatment device.

FIG. 5 depicts an example of assembling a monitoring and treatment device.

FIG. 6 depicts another example of assembling a monitoring and treatment device.

FIG. 7 depicts another example of assembling a monitoring and treatment device.

FIG. 8 depicts another example of assembling a monitoring and treatment device.

FIG. 9 depicts another example of assembling a monitoring and treatment device.

FIG. 10 depicts another example of assembling a monitoring and treatment device.

FIG. 11 depicts an example of predetermined anatomical regions for components of a monitoring and treatment device.

FIG. 12 depicts another example of cardioprotective apparel for a monitoring and treatment device.

FIG. 13A depicts additional views of the cardioprotective apparel of FIG. 12.

FIG. 13B depicts another example of cardioprotective apparel for a monitoring and treatment device.

FIG. 13C depicts another example of cardioprotective apparel for a monitoring and treatment device.

FIG. 14 depicts another example of cardioprotective apparel for a monitoring and treatment device.

FIG. 15A depicts additional views of the cardioprotective apparel of FIG. 14.

FIG. 15B depicts another example of cardioprotective apparel for a monitoring and treatment device.

FIG. 15C depicts another example of cardioprotective apparel for a monitoring and treatment device.

FIG. 15D depicts an additional view of the cardioprotective apparel of FIG. 15C.

FIG. 16A depicts another example of cardioprotective apparel for a monitoring and treatment device.

FIG. 16B depicts another example of cardioprotective apparel for a monitoring and treatment device.

FIG. 16C depicts another example of cardioprotective apparel for a monitoring and treatment device.

FIG. 16D depicts an additional view of the cardioprotective apparel of FIG. 16C.

FIG. 16E depicts an additional view of the cardioprotective apparel of FIGS. 16C and 16D.

FIG. 16F depicts an additional view of the cardioprotective apparel of FIGS. 16C-16E.

FIG. 17 depicts an example of cardioprotective apparel including material zones.

FIG. 18 depicts an example electronic architecture for a cardiac device controller.

FIG. 19 depicts an example monitoring and treatment device with an assemblable gel pack.

### DETAILED DESCRIPTION

Wearable cardiac devices implementing the devices, systems, methods, and techniques disclosed herein, such as wearable cardiac treatment devices, can be used in clinical care settings to monitor for treatable cardiac arrhythmias and provide treatments such as defibrillation, cardioversion, or pacing shocks in the event of life-threatening arrhythmias. Thus, while the patient is wearing the wearable cardiac device, the device is configured to detect and treat these life-threatening arrhythmias. The wearable cardiac device may also provide alarms to the patient, warning the patient of an impending shock that the patient may be able to delay or cancel by pressing one or more response buttons thereby indicating that the patient is still conscious.

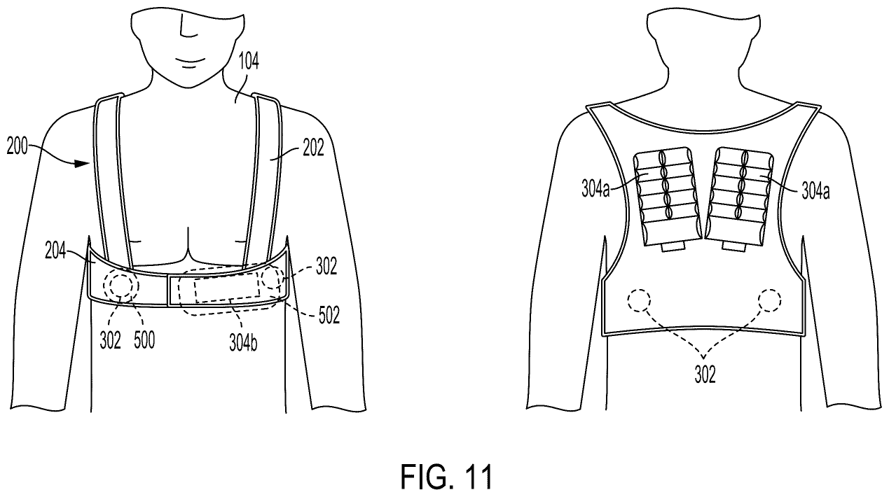

Such a monitoring and treatment device configured for an ambulatory patient may include cardioprotective apparel configured to be worn by a patient, such as around an upper torso region of the patient. Various components of the monitoring and treatment device, such as ECG sensing electrodes and therapy electrodes, may need to be assembled into the cardioprotective apparel before the patient can wear and use the monitoring and treatment device. Alternatively, or additionally, at least some of the components of the monitoring and treatment device may be permanently disposed (e.g., permanently sewn, adhered, etc.) into the cardioactive apparel. The components may need to be supported in a particular way once the cardioactive apparel is donned by the patient. For example, the sensing electrodes may need close and continuous contact with the patient's skin in order to sense the patient's cardiac activity and generate ECG signals from the sensed cardiac activity. Additionally, the sensing electrodes may need to remain in certain general anatomical regions on the patient in order to properly sense the patient's cardiac activity. As another example, the therapy electrodes may similarly need to remain on or near certain general anatomical regions on the patient in order to properly deliver therapeutic pulses to the patient if the monitoring and treatment device detects that the patient is experiencing a treatable arrhythmia. Examples of such anatomical regions are shown and described with respect to FIG. 11 below.

In order to serve the maximum patient population, the monitoring and treatment device needs to be configured such it can be worn by patients of all body types and shapes. For patients with larger breast tissue, such as female patients or male patients in larger sizes, the breast tissue may interfere with the proper placement of the components of the monitoring and treatment device with respect to the patient's anatomy. This interference may especially affect the sensing and/or therapy electrodes configured to be placed against or near the patient's front (e.g., the patient's anterior torso anatomy). Other anatomical differences between patients, especially for patients in larger sizes, may also interfere with the proper placement of the system components with respect to the patient's anatomy. For example, such anatomical differences may include front torso projections due to fat deposits in patients.

As such, it may be beneficial for such monitoring and treatment devices to include mechanical accommodations that are configured to shape and/or shift patient tissue such that proper system component, particularly electrode placement, occurs when the monitoring and treatment device is worn by the patient. This disclosure thus relates to an ambulatory patient monitoring and treatment device configured to be worn continuously by a patient. The ambulatory patient monitoring and treatment device includes ECG electrodes configured to sense cardiac activity of the patient, therapy electrodes configured to deliver one or more electrical therapeutic shocks to the patient, and cardioprotective apparel configured to be donned about an upper torso region of the patient. The monitoring and treatment device also includes a device controller and associated circuitry operably connected to the ECG sensing electrodes and therapy electrodes. Using the sensed cardiac activity from the ECG sensing electrodes, the device controller is configured to determine whether the patient is experiencing a treatable arrhythmia. The device controller is further configured to, in response to determining that the patient is experiencing a treatable arrhythmia, instruct the delivery of the one or more electrical therapeutic shocks to the patient via the therapy electrodes.

In implementations, the cardioprotective apparel includes a cardioactive band configured to be disposed on an inferior end of the upper torso region of the patient (e.g., in the region under the patient's breast tissue and near the tip of the patient's sternum) and at least two shoulder harnesses attached to the cardioactive band and configured to support the cardioactive apparel on the patient's shoulders. Additionally, the cardioactive band is configured to support (1) at least one anterior therapy electrode and (2) a number of ECG sensing electrodes distributed in a spaced apart configuration. When the monitoring and treatment device is worn by the patient, the cardioactive band should (1) support the at least one anterior therapy electrode such that the at least one anterior therapy electrode is disposed on an anterior therapy electrode placement region and (2) support the ECG sensing electrodes such that the ECG sensing electrodes are disposed on predetermined ECG sensing anatomical regions when the monitoring and treatment device is worn by the patient.

However, to facilitate the cardioactive band in supporting the at least one anterior therapy electrode and ECG electrodes in the correct regions when the monitoring and treatment device is worn by the patient, each of the at least two shoulder harnesses is configured to shift and/or shape breast tissue of the patient's upper torso region. For example, the at least two shoulder harnesses may shift the breast tissue away from the anterior therapy electrode placement region and/or at least one of the predetermined ECG sensing anatomical regions. As an illustration, the at least two shoulder harnesses may each include a sling portion positioned around the junction of the shoulder harness and the cardioactive band. Each sling portion shifts the patient's breast tissue away from where the anterior therapy electrode and the ECG sensing electrodes configured to be positioned on the patient's anterior anatomy sit on or near the patient's skin. The result of the shifting of the patient's breast tissue is such that the at least one anterior therapy electrode and the ECG sensing electrodes lie in a flush manner with the patient's skin when the monitoring and treatment device is worn by the patient. That the electrodes lie flush against the patient's skin helps the electrodes function properly while the patient is wearing and using the monitoring and treatment device, particularly when the patient is moving.

For instance, when the therapy electrodes sit flush with the patient's skin, the therapy electrodes can deliver therapy energy in the correct vectors and transfer the maximum amount of energy to the patient in case the device controller detects that the patient is experiencing a treatable arrhythmia. As another example, the ECG sensing electrodes may need good contact with the patient's skin to sense cardiac activity of the patient. Accordingly, when the shoulder harnesses shift the patient's breast tissue away from the predetermined ECG sensing anatomical regions such that the ECG sensing electrodes can lie flush with the patient's skin, the ECG sensing electrodes can better sense the patient's cardiac activity and generate ECG signals.

Additionally, the shoulder harnesses may also function to make it more comfortable for the patient to wear a bra or other supportive garment with the monitoring and treatment device. For example, the shoulder harnesses maybe configured to lie on the outside of the patient's shoulder (e.g., a lateral portion of the patient's shoulder) such that the shoulder harnesses and the straps of the patient's bra do not overlap. As another example, the shoulder harnesses may be configured to adjustment mechanisms in the base of the shoulder harness (e.g., near the junction with the cardioactive band), or the cardioactive band may include the adjustment mechanisms, to avoid overlap between strap adjustment mechanisms for the bra and the adjustment mechanisms for the shoulder harnesses. As another example, the shoulder harnesses may include a sling portion that functions as described above and further works to stabilize the patient's breast tissue within a bra worn over the cardioprotective apparel rather than fighting the forces exerted by the bra by displacing the breast tissue to the side.

In implementations, the monitoring and treatment device may include additional features that allow for a better fit with the patient's anatomy and thus facilitate the electrodes with lying in a flush manner with the patient's skin when the patient wears the monitoring and treatment device. To illustrate, in implementations, the monitoring and treatment device may include one or more modifications to accommodate front torso projections, especially in larger sizes for the cardioprotective apparel. Such modifications may include, for instance, contouring according to male versus female anatomy of the front torso, thicker cardioactive bands in larger sizes of the cardioprotective apparel, tapered and/or conical clasps in larger sizes of the cardioprotective apparel to better accommodate the front torso and prevent pinching or pressure points, cardioprotective apparel with zones having different elasticities to better conform to patient anatomy, and/or the like. As another illustration, the cardioprotective apparel may be configured in gendered sizes that are shaped to conform to female anatomy versus male anatomy. These modifications for accommodating front torso projections may further help, for instance, therapy electrode(s) and/or ECG sensing electrode(s) lie flush with the patient's skin and prevent flipping of portions or all of the cardioactive band. For example, the cardioprotective apparel may be configured to have a unisex set of smaller sizes. The sizes of the cardioprotective apparel may then split into male sizes and female sizes beyond a certain size point, where the male sizes include measurements, elasticity zones, clasp shapes, etc. that are tailored to male anatomy in larger sizes and where the female sizes include measurements, elasticity zones, clasp shapes, etc. that are tailored to female anatomy in larger sizes.

In one example use case, a cardiologist may prescribe that a female patient with a risk for developing a life-threatening arrhythmia use an ambulatory monitoring and treatment device until the patient can receive an implantable defibrillator. The ambulatory monitoring and treatment device includes cardioprotective apparel having shoulder harnesses and a cardioactive band that the patient wears around their torso. As part of receiving the ambulatory monitoring and treatment device, the patient consults with a technician who fits the patient for the cardioprotective apparel and determines which size of the cardioprotective apparel the patient should receive. The fit of the cardioprotective apparel should be such that both the therapy electrodes and the ECG sensing electrodes are positioned near or against the patient's skin in particular anatomical regions. Additionally, the shoulder harnesses of the cardioactive band each include sling portions that shift the patient's breast tissue away from the particular anatomical regions on the patient's anterior. This shifting of the patient's breast tissue helps the therapy electrode(s) and the ECG sensing electrode(s) better lie flush with particular anatomical regions on the patient's torso. The therapy electrodes are thus able to provide better therapy to the patient, and the ECG sensing electrode(s) more clearly sense surface electrical signals from the patient (e.g., with less interference or noise).

Additionally, the cardioprotective apparel is configured to increase comfort and functionality for the monitoring and treatment device while being worn concurrently with a bra. For example, the cardioprotective apparel includes wide shoulder harnesses configured to sit on lateral portions of the patient's shoulders, as well as a strap adjustment mechanism at the front base of the shoulder harnesses. These configurations may help minimize the overlap between the straps of the patient's bra and the shoulder harnesses. The sling portion is also configured to stabilize the breast tissue within the bra when both the monitoring and treatment device and the bra are worn by the patient.

In another example use case, a cardiologist may determine that a patient is as risk of heart failure (e.g., as a result of a cardiac episode, such as a myocardial infarction) and prescribe that the patient use an ambulatory monitoring and treatment device while the patient completes a cardiac rehabilitation plan. The patient may wear the monitoring and treatment device during their day-to-day life, which includes performing exercises as part of the cardiac rehabilitation plan. The monitoring and treatment device includes shoulder harnesses configured to shape and shift the patient's upper torso anatomy, including the patient's breast tissue, away from locations on the patient's anterior anatomy where therapy electrode(s) and/or ECG sensing electrode(s) need to sit to properly protect the patient from life-threatening arrhythmias as described above. Additionally, cardioprotective apparel of the monitoring and treatment device includes features to accommodate front torso projections of the patient, including a wider band than in smaller cardioprotective apparel sizes, a tapered clasp, and zones with different elasticity zones to accommodate the patient's anatomy according to their sex. These additional features of the cardioprotective apparel further help the therapy electrode(s) and/or ECG sensing electrode(s) properly sit against or near the patient's anatomy.

The ambulatory patient monitoring and treatment devices described herein may provide advantages over prior art systems. For one, a patient monitoring and treatment device including cardioprotective apparel where the cardioprotective apparel has shoulder straps that shape and/or shift patient breast tissue as described above help the electrodes lie flush with the patient's skin. In turn, as also described above, this helps the electrodes function more properly. For example, therapy electrodes can more effectively deliver therapeutic energy to the patient by making better direct or indirect (e.g., via a conductive mesh lying between the therapy electrode and the patient's skin) skin contact and in the correct, predetermined vectors. As another example, ECG sensing electrodes can better and more clearly (e.g., with less noise) sense cardiac activity of the patient when the ECG sensing electrodes lie flush against the patient's skin. These shoulder straps that shape and/or shift the patient's breast tissue may also help other components of the monitoring and treatment device remain in correct, predetermined locations. For instance, the configuration of the shoulder straps described herein can help keep the shoulder straps lie more flatly against the patient's skin. Thus, as an example, a user interface including one or more buttons that the patient can press to suspend therapeutic shocks to the patient that is positioned on one of the shoulder straps may more easily remain in the predetermined position on the shoulder strap, which is also a more comfortable position that is also easier for the patient to access.

Similar advantages may be conferred by other features of the cardioprotective apparel that help the monitoring and treatment device lie more flush with or flat against the patient's skin. Such other types of features may include, among others, wider cardioactive bands for larger sides, shoulder harnesses configured to lie laterally on the patient's shoulders, contoured cardioactive bands for male and female anatomy, zones with different elasticities to accommodate patient anatomy according to different sizes, tapered closures for the cardioprotective apparel, and/or combinations of such features.

In addition, a patient monitoring and treatment device including cardioprotective apparel where the cardioprotective apparel has shoulder straps that shape and/or shift patient breast tissue as described above may also help the cardioprotective apparel to be more effectively and more comfortably worn with a bra. For example, as discussed above, the placement of the shoulder harnesses on the patient's shoulders and/or the placement of adjustment mechanisms for the shoulder harnesses may make the cardioprotective apparel more comfortable to be worn with a bra by minimizing interference or overlap between the cardioprotective apparel and the bra. As another example, sling portions of the shoulder straps may work to stabilize breast tissue within the bra while shifting the breast tissue away from anatomical regions that the electrodes need to lie near or against. By making the cardioprotective apparel easier and more comfortable to wear with a bra, patients may be more likely to comply with a prescription to wear the monitoring and treatment device. Moreover, in at least some cases, wearing the bra with the monitoring and treatment device may increase the effectiveness of the monitoring and treatment device. To illustrate, clastic elements of the bra may help keep one or more therapy electrodes and/or one or more ECG sensing electrodes supported by the cardioactive band of the cardioprotective apparel more firmly near or against predetermined anatomical regions of the patient.

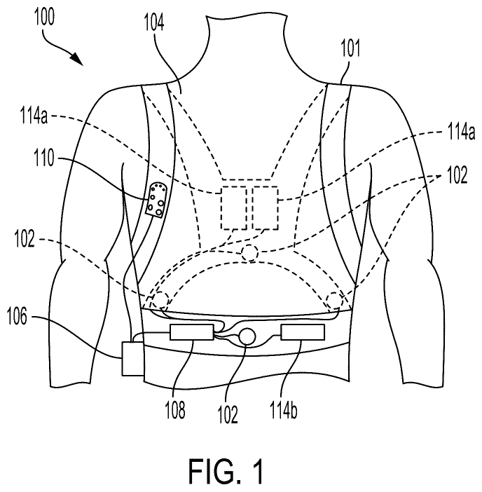

FIG. 1 illustrates an example of an ambulatory monitoring and treatment device 100, according to implementations disclosed herein. As shown in FIG. 1, the wearable cardiac device 100 is external and wearable by a patient 104 around the patient's torso. Such a wearable cardiac device 100 can be, for example, capable and designed for moving with the patient 104 as the patient 104 goes about their daily routine. For instance, the wearable cardiac device 100 may be configured to be bodily-attached to the patient 104. As noted above, the wearable cardiac device 100 may be a wearable defibrillator or a wearable cardioverter defibrillator. In one example scenario, such wearable defibrillators can be worn nearly continuously or substantially continuously for an extended period of time, such as a week, two weeks, a month, two months, three months, six months, etc. at a time. During the period of time in which they are worn by the patient 104, the wearable defibrillators can be configured to continuously or substantially continuously monitor the vital signs of the patient 104 and can be configured to, upon determination that treatment is required, deliver one or more therapeutic electrical pulses to the patient 104. For example, such therapeutic shocks can be pacing, defibrillation, cardioversion, and/or transcutaneous electrical nerve stimulation (TENS) pulses.

As shown in FIG. 1, the ambulatory patient monitoring and treatment device 100 can include cardioprotective apparel 101 configured to be donned about an upper torso region of the patient, one or more ECG sensing electrodes 102 configured to sense cardiac activity of the patient 104, and one or more therapy electrodes 114a and 114b (collectively referred to herein as therapy electrodes 114) configured to deliver one or more electrical therapeutic shocks. In implementations, the ambulatory patient monitoring and treatment device 100 may further include other elements, such as a signal processing unit 108 (e.g., a connection pod), a patient interface pod 110, or any combination of these. In implementations, the monitoring and treatment device 100 may also include additional sensors, such as one or more motion detectors configured to generate motion data indicative of physical activity performed by the patient 104, one or more wear state sensors configured to detect a wear state of the wearable defibrillator 100, one or more vibrational or bioacoustics sensors configured to generate bioacoustics signals for the heart of the patient 104, one or more respiration sensors configured to generate respiration signals indicative of the respiration activity of the patient 104, and/or the like.

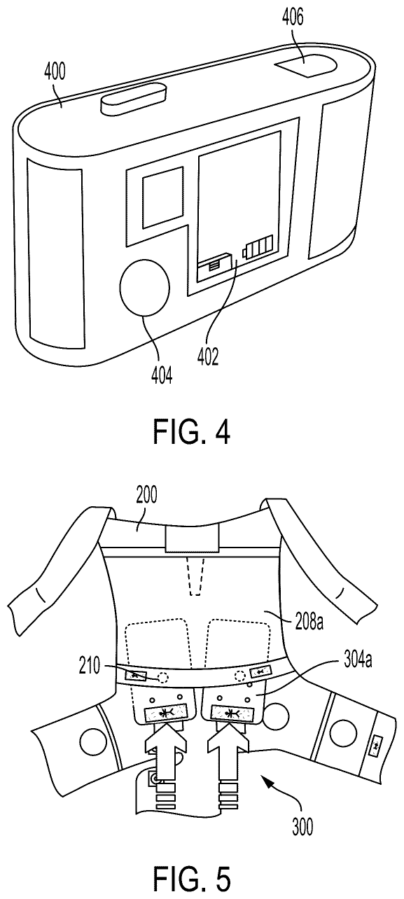

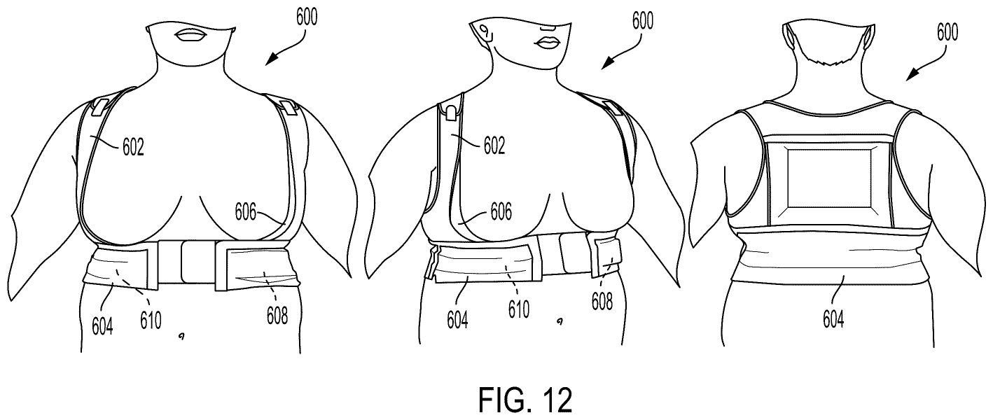

Some or all of the one or more sensing electrodes 102, the one or more therapy electrodes 114, the signal processing unit 108, patient interface pod 110, etc. may be configured to be assembled into the cardioprotective apparel 101, as described in further detail below with respect to FIGS. 12-16F. For example, at least some of the components of the wearable defibrillator 100 can be configured to be disposed on the cardioprotective apparel 101 by being removably mounted on or affixed to the cardioprotective apparel 101, such as by mating hooks, hook-and-loop fabric strips, receptacles (e.g., pockets), snaps (e.g., plastic or metal snaps), and the like. For instance, the sensing electrodes 102 may be removably attached to the cardioprotective apparel 101 by hook-and-loop fabric strips on the sensing electrodes 102 and the cardioprotective apparel 101, and the therapy electrodes 114 may be removably attached on the cardioprotective apparel 101 by being inserted into receptacles of the cardioprotective apparel 101. In examples, elements that can be physically assembled into the cardioprotective apparel 101 are physically coupled to each other as an electrode belt (e.g., as shown by electrode belt 300 of FIG. 3). In examples, the electrode belt is coupled to a cardiac device controller 106 and associated circuitry via a connector (e.g., via connector 308 of FIG. 3). In some examples, at least some of the components of the monitoring and treatment device 100 can be permanently integrated into the cardioprotective apparel 101, such as by being sewn into the garment or by being adhesively secured to the cardioprotective apparel 101 with a permanent adhesive. In examples, at least some of the components may be connected to each other through cables, through sewn-in connections (e.g., wires woven into the fabric of the cardioprotective apparel 101), through conductive fabric of the cardioprotective apparel 101, and/or the like.

As noted above, the cardiac device controller 106 and associated circuitry can be operatively coupled to the ECG electrodes 102 and the therapy electrodes 114, which can be temporarily or removably affixed to the cardioprotective apparel 101 (e.g., assembled into the cardioprotective apparel 101 or removably attached to the cardioprotective apparel 101, for example, using hook-and-loop fasteners) and/or permanently integrated into the cardioprotective apparel 101 as discussed above. As shown in FIG. 1, the ECG electrodes 102 and/or the therapy electrodes 114 can be directly operatively coupled to the cardiac controller 106 and/or operatively coupled to the cardiac controller 106 through another component of the monitoring and treatment system 100, such as the signal processing unit 108. Component configurations other than those shown in FIG. 1 are also possible. For example, the ECG electrodes 102 can be configured to be attached at various positions about the body of the patient 104.

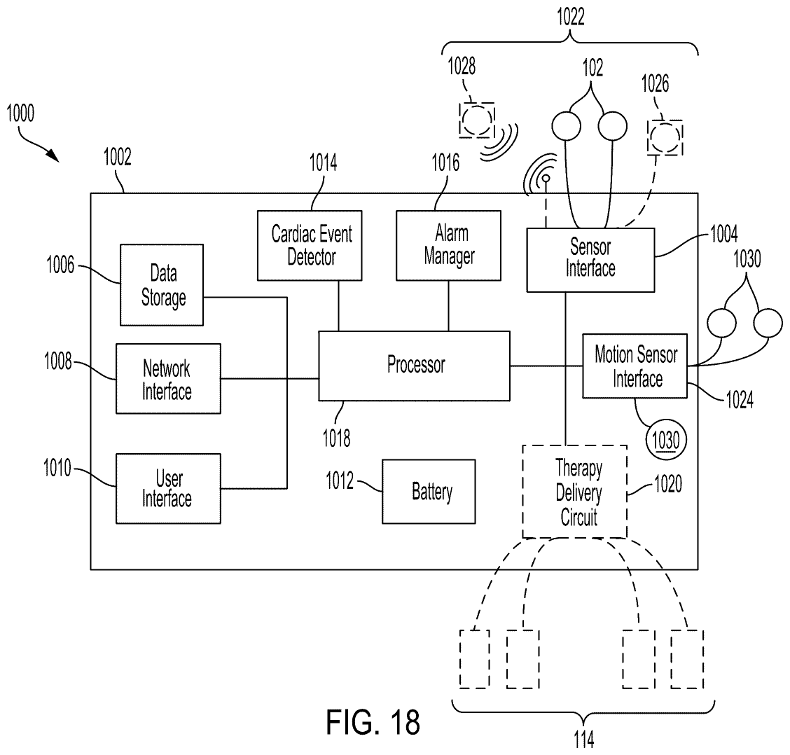

In some implementations, the cardiac device controller 106 may also be configured to be assembled into the cardioprotective apparel 101. For example, the entire cardiac controller 106 as shown in FIG. 1 may be configured to be inserted into or attached to a receptacle of the cardioprotective apparel 101, such as a pocket. As another example, the functions of the cardiac controller 106 may be dispersed among multiple cardiac controller units (e.g., a cardiac arrhythmia monitoring unit, a therapy delivery unit, a communications unit, an alarm module unit, etc., such as discrete units for two or more of the components shown in the electronic architecture of FIG. 18). These multiple cardiac controller units may then be inserted into and/or attached to receptacles of the cardioprotective apparel 101.

As discussed above, the sensing electrodes 102 can be configured to sense cardiac activity of the patient 104. Example sensing electrodes 102 may include a metal electrode with an oxide coating such as tantalum pentoxide electrodes. For example, by design, the sensing electrodes 102 can include skin-contacting electrode surfaces that may be deemed polarizable or non-polarizable depending on a variety of factors including the metals and/or coatings used in constructing the electrode surface. All such electrodes can be used with the principles, techniques, devices, and systems described herein. For instance, the electrode surfaces can be based on stainless steel, noble metals such as platinum, or Ag—AgCl.

In implementations, the sensing electrodes 102 can be used with an electrolytic gel dispersed between the electrode surface and the patient's skin. In implementations, the sensing electrodes 102 can be dry electrodes that do not need an electrolytic material. As an example, such a dry electrode can be based on tantalum metal and having a tantalum pentoxide coating as is described above. Such dry electrodes can be more comfortable for long-term monitoring applications.

In implementations, the sensing electrodes 102 can include additional components such as accelerometers, acoustic signal detecting devices (e.g., vibrational sensors), and other measuring devices for recording additional parameters. For example, the sensing electrodes 102 can also be configured to detect other types of patient physiological parameters and acoustic signals, such as tissue fluid levels, heart vibrations, lung vibrations, respiration vibrations, patient movement, etc. In some examples, the therapy electrodes 114 can additionally or alternatively be configured to include sensors configured to detect ECG signals as well as, or in the alternative to, other physiological signals from the patient 104. In accordance with the principles of this disclosure, such biophysical or physiological sensors can implement devices, systems, methods, and techniques described herein to facilitate assembly of such biophysical or physiological sensors into the cardioprotective apparel 101, and/or continuous or intermittent monitoring of the proper location or position of the biophysical or physiological sensors.

The signal processing unit 108 can, in some examples, include a signal processor configured to amplify, filter, and digitize cardiac signals, such as the ECG signals generated from the sensed cardiac activity of the patient 104, prior to transmitting the cardiac signals to the cardiac controller 106. As an example, the signal processing unit 310 may be configured to reduce and/or remove noise in the signals received by the sensing electrodes 302 (e.g., by using signals from a ground electrode, which may be one of the sensing electrodes 302, one of the therapeutic electrodes 304, and/or may be provided elsewhere on the wearable cardiac device). As another example, the signal processing unit 310 may be configured to digitize the signals received by the sensing electrodes 302 (e.g., by an analog-to-digital converter).

In examples, the signal processing unit 108 may be located on the small of the patient's back, as to on the patient's front as illustrated in FIG. 1. In such implementations, because the signal processing unit 108 is located on the small of the patient's back where the patient may be sensitive to feeling movement, the signal processing unit 108 can be configured to include one or more vibration motors to provide tactile notifications to the patient. For instance, the signal processing unit 108 can receive one or more signals from the cardiac controller 106 and provide a tactile alert to the patient 104 based on the one or more signals from the cardiac controller 106.

The one or more therapy electrodes 114 can be configured to deliver one or more electrical therapeutic shocks to the patient 104, such as one or more therapeutic cardioversion/defibrillation shocks to the body of the patient 104, when the wearable cardiac device 100 determines that such treatment is warranted based on the signals detected by the sensing electrodes 102 and processed by the cardiac controller 106. Example therapy electrodes 114 can include conductive metal electrodes such as stainless-steel electrodes that include, in certain implementations, one or more conductive gel deployment devices configured to deliver conductive gel between the metal electrode and the patient's skin prior to delivery of a therapeutic shock.

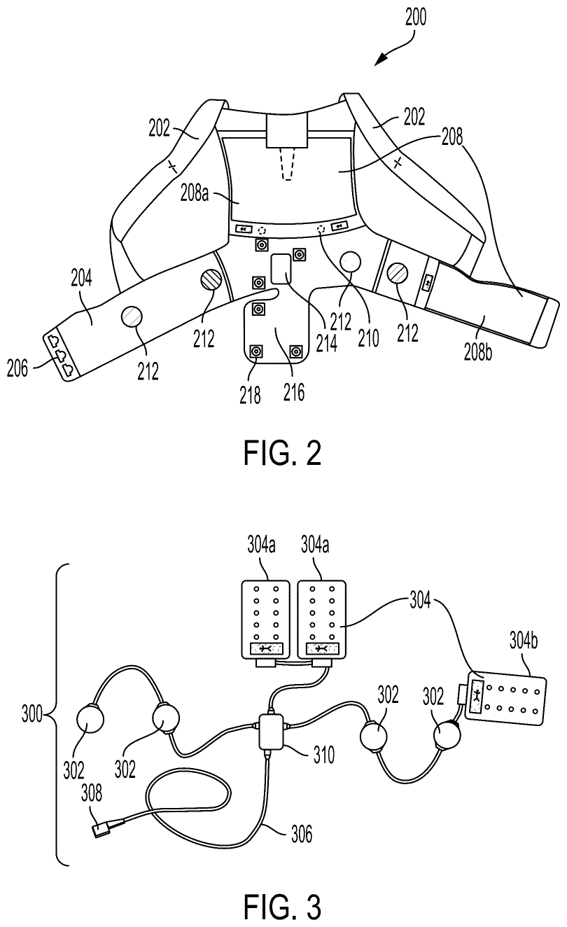

Accordingly, in implementations, the cardiac device controller 106 is configured to determine whether the patient is experiencing a treatable arrhythmia based on the sensed cardiac activity and instruct the delivery of one or more electrical therapeutic shocks to the patient via the one or more therapy electrodes 114 in response to determining that the patient 104 is experiencing a treatable arrhythmia. The functionality of the cardiac device controller 106 is described in further detail below with respect to FIG. 18. In implementations, the cardiac device controller 106 further warns the patient 104 prior to the delivery of a therapeutic shock, such as via output devices integrated into or connected to the cardiac device controller 106, the signal processing unit 108, and/or the patient interface pod 110. In examples, the patient interface pod 110 can be removably attached to or disposed on the cardioprotective apparel 101. For example, the patient interface pod 110 can be secured to a hook-and-loop fastener and/or a plastic or metal snap connector disposed on the shoulder strap of the cardioprotective apparel 101 (e.g., shoulder harness 202 shown in FIG. 2). The warning, for example, may be auditory (e.g., a siren alarm, a voice instruction indicating that the patient 104 is going to be shocked), visual (e.g., flashing lights on the cardiac device controller 106), haptic (e.g., a tactile, buzzing alarm generated by the signal processing unit 108), and/or the like. If the patient 104 is still conscious, the patient 104 may be able to delay or stop the delivery of the therapeutic shock. For example, the patient 104 may press one or more buttons on the patient interface pod 110 to indicate that the patient 104 is still conscious. In response to the patient 104 pushing the one or more buttons, the cardiac controller 106 may delay or stop the delivery of the therapeutic shock.

Additionally or alternatively, in implementations, the signal processing unit 108 may be configured control at least part of the delivery of therapeutic pulses via the therapy electrodes 114. As an example, the signal processing unit 108 may receive a signal from the device controller 106 initiating a therapy delivery sequence. The signal processing unit 108 may send a signal to the therapy electrodes 114 to activate the deployment of conductive electrolytic gel at the therapy electrodes 114 (e.g., from a permanent or removable gel pack incorporated as part of the therapy electrodes 114, from a removable gel pack configured as an assemblable element, such as removable gel pack 244 shown in FIG. 26). The signal processing unit 108 may then receive one or more therapeutic charges from the controller 400 and convey the one or more therapeutic charges to the therapy electrodes 114 for delivery of one or more therapeutic pulses to the patient 104.

Example implementations of a wearable cardiac device in accordance with the devices, systems, techniques, and methods disclosed herein are shown in FIGS. 2-4. FIG. 2 illustrates an embodiment of cardioprotective apparel 200 configured to be worn by a patient for an extended period of time and for receiving components of the monitoring and treatment device 100 that are assemblable into the cardioprotective apparel 200, as described herein. As shown in FIG. 2, the cardioprotective apparel 200 includes at least two shoulder harnesses 202 configured to be worn over the patient's shoulders and a cardioactive band 204 configured to wrap around the patient's torso. The cardioactive band 204 is configured to be disposed on an inferior end of the upper torso region of the patient 104, as further illustrated in FIG. 2, and support active monitoring and/or treatment components of the monitoring and treatment device 100. For example, as described in more detail below, the cardioactive band 204 may support at least one therapy electrode 114 and at least one sensing electrode 102. The at least two shoulder harnesses 202 are attached to the cardioactive band 204 and support the cardioprotective apparel 200 on the patient's shoulders.

In the example of FIGS. 2-4, once on the patient 104, the cardioprotective apparel 200 may be fixed in place with a clasp 206, such as a clasp 206 provided on the cardioactive band 204 as illustrated (e.g., in order to secure the cardioprotective apparel 200 and the assembled and/or permanently integrated components against the body of the patient 104). For instance, the clasp 206 may include a first clasp portion located at one end of the cardioactive band 204 as shown in FIG. 2 configured to removably attach to or mate with a second clasp portion located at the other end of the cardioactive band 204 (e.g., on the side of the cardioprotective apparel 200 not shown in FIG. 2, the side opposite from the pocket 208 on the cardioactive band 204). As such, the clasp 206 may include mating hooks and eyes, hook-and-loop fabric strips, buttons, snaps, and/or the like. In implementations, the clasp 206 may be configured with multiple settings such that the patient 104 can adjust the tightness of fit of the cardioprotective apparel 200 and/or the positioning or force applied to some or all of the assembled and/or permanently integrated components against the body of the patient 104. To illustrate, as an example, the clasp 206 may include a row of hooks provided on one end of the band 204 and several rows of eyes provided on one the other end of the cardioactive band 204, with each row of eyes a spaced distanced from the others. The patient 104 can then mate the hooks to the row of eyes that provides for the best fit of the cardioactive band 204 (and the assembled and/or permanently integrated components) on the patient 104. As another example, the clasp 206 may include a strip of hook fabric on one end of the cardioactive band 204 and a longer strip of loop fabric on the other end of the cardioactive band 204 such that the patient 104 can attach the hook fabric to a spot on the loop fabric that provides for the best fit of the cardioactive band 204 on the patient 104.

As noted, in implementations, the cardioprotective apparel 200 is configured to receive various components of the monitoring and treatment device 100 that are assemblable into the cardioprotective apparel 200. Accordingly, the cardioprotective apparel 200 may be configured to receive assemblable elements or components of an electrode belt, such as the electrode belt 300 shown in FIG. 3, before the cardioprotective apparel 200 is worn by the patient 104. In implementations, the electrode belt 300 is configured to facilitate sensing electrical signals associated with cardiac activity of the patient 104 and/or deliver one or more therapeutic pulses to the patient 104. Accordingly, as illustrated in FIG. 3, the electrode belt 300 may include at least one sensing electrode 302 configured to sense electrical signals associated with cardiac activity of the patient 104 (e.g., configured similarly to the sensing electrodes 102 discussed with reference to FIG. 1) and/or at least one therapeutic electrode 304 configured to deliver one or more therapeutic pulses to the patient 104 (e.g., configured similarly to the therapy electrodes 114 discussed with reference to FIG. 1).

In implementations, the assemblable components of the electrode belt 300 may include both the sensing electrodes 302 and the therapeutic electrodes 304, as shown in FIG. 3. In other implementations, the assemblable components of the electrode belt 300 may include only sensing electrodes 302 or only therapeutic electrodes 304. For example, the assemblable elements of the electrode belt 300 may include only therapeutic electrodes 304, and the cardioprotective apparel 200 may instead be provided with sensing electrodes that are already permanently integrated into the cardioprotective apparel 200. Such permanently integrated sensing electrodes may be, for instance, sensing electrode discs that are sewn or permanently adhered to the cardioprotective apparel 200 or conductive thread sewn into the cardioprotective apparel 200.

In implementations, the assemblable components of the electrode belt 300 may include only sensing electrodes 302, and the cardioprotective apparel 200 may be provided with therapy electrodes permanently integrated into the cardioprotective apparel 200. To illustrate, the cardioprotective apparel 200 may include therapy electrode pads sewn or permanently adhered to the cardioprotective apparel 200. In such implementation, the cardioprotective apparel 200 may include pockets or other compartments for replaceable gel packs provided with electrolytic gel and disposed proximate the permanently integrated therapy electrodes. In these implementations, the assemblable components of the monitoring and treatment system 100 include the replaceable gel packs provided with the electrolytic gel and disposed proximate the permanently integrated therapy electrodes. The gel packs may be configured to deploy conductive gel in the interface between the permanently integrated therapy electrodes and the patient's skin in advance of a therapeutic shock, as discussed above with reference to FIG. 1.

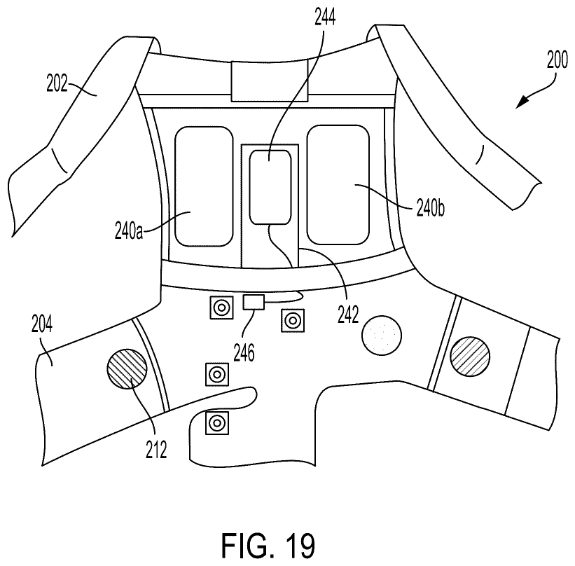

An example of the cardioprotective apparel 200 including permanently integrated therapy electrodes is shown in FIG. 19. In the example illustrated in FIG. 19, the back side of the cardioprotective apparel 200 is provided with therapy electrode pads 240a and 240b (collectively 240) permanently disposed on the cardioprotective apparel 200. For example, therapy electrodes pads 240 may be configured similarly to the therapeutic electrodes sewn into the cardioprotective apparel 200 or permanently adhered to the cardioprotective apparel 200. The back side of the cardioprotective apparel 200 also includes a gel pack pocket 242 configured to receive a replaceable gel pack 244. In implementations, the replaceable gel pack 244 is configured to disposed next to the permanently integrated therapy electrode pads 240a and 240b such that the replaceable gel pack 244 can disperse conductive gel into the interface between the therapy electrode pads 240 and the patient's skin. In implementations, the cardioprotective apparel 200 may include multiple gel pack pockets 242 configured to receive multiple replaceable gel packs 244. For instance, the cardioprotective apparel 200 may include two gel pack pockets 242, each disposed next to a therapy electrode pad 240 and each configured to receive a replaceable gel pack 244.

The electrode belt 300 includes wires 306 connecting the assemblable elements of the electrode belt 300 to each other and, ultimately, to a connector 308. In turn, the connector 308 is configured to removably attach to a controller configured to monitor and treat the patient 104, such as the controller 400 shown in FIG. 4. The controller 400 may be configured similarly to the cardiac controller 106 described with reference to FIG. 1. Additional details about implementations of the controller 400 are also provided below with reference to FIG. 18. The connector 308 may be configured to connect into a mating slot on the controller 400 as shown in FIG. 4 such that the controller 400 can receive signals from (e.g., signals containing information about cardiac electrical activity sensed from the patient 104) and transmit signals to (e.g., signals instructing the delivery of one or more therapeutic pulses) to the electrode belt 300. The controller 400 is configured to be worn by the patient 104, such as within a holster with a shoulder harness. Alternatively, as described above with reference to the cardiac controller 106 of FIG. 1, in implementations the controller 400 may be configured as one or more assemblable components also configured to be assembled into the cardioprotective apparel 200.

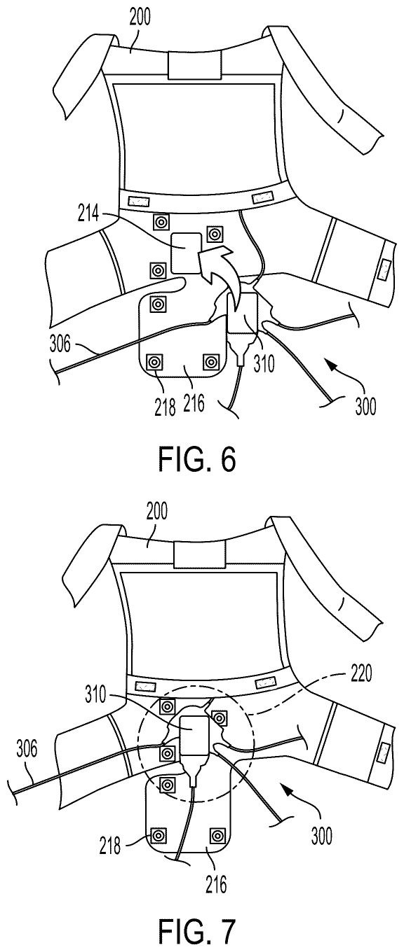

Returning to FIG. 3, in implementations, the electrode belt 300 includes one or more assemblable elements in addition to the sensing electrodes 302 and/or therapeutic electrodes 304. For example, as shown in FIG. 3, the electrode belt 300 may include a signal processing unit 310 configured to electrically connect at least one electrode to the controller 400. For instance, the signal processing unit 310 may connect the sensing electrodes 302 and/or the therapeutic electrodes 304 to the controller 400 via the wires 306 and the connector 308 when the connector 308 is mated to the controller 400. In some implementations, the signal processing unit 310 may be configured to connect both the sensing electrodes 302 and the therapeutic electrodes 304 to the controller 400, as illustrated in FIG. 3. In some implementations, the signal processing unit 310 may be configured to only connect the sensing electrodes 302 or only the therapeutic electrodes 304 to the controller 400. For example, the electrode belt 300 may be configured differently from the electrode belt 300 shown in FIG. 3 (e.g., with the signal processing unit 310 provided between either the sensing electrodes 302 or the therapeutic electrodes 304 but not both), and/or the electrode belt 300 may not include the sensing electrodes 302 or the therapeutic electrodes 304. In embodiments, other configurations of the electrode belt 300 may alternatively be used, such as the version of the electrode belt 300 shown in FIGS. 6 and 7 with a differently implemented signal processing unit 310. As another example, some implementations of the electrode belt 300 may not include a signal processing unit 310, with the functions of the signal processing unit 310 being performed at the controller 400, the sensing electrodes 302, and/or the therapeutic electrodes 304.

An example of a controller 400 connected to the sensing electrode 302 and therapeutic electrodes 304 (and/or other components of the monitoring and treatment device 100) is shown in FIG. 4. As noted above, in implementations, the controller 400 is configured similarly to the device controller 106 shown and described above with respect to FIG. 1 and further with respect to FIG. 18 below. In implementations, the controller 400 is configured to generate an ECG signal based on the electrical signals sensed by the sensing electrodes 302 and determine whether the patient 104 is experiencing a treatable cardiac arrhythmia using the ECG signal. If the controller 400 determines that the patient 104 is experiencing a treatable cardiac arrhythmia, the controller 400 may further generate one or more therapeutic pulses for delivery to the patient 104 via the at least one therapeutic electrode 114. Before generating the one or more therapeutic pulses, however, the controller 400 may activate one or more alarms for a predetermined alarm period to warn the patient 104 that a therapeutic shock is imminent. The alarms may be delivered to the patient 104 via one or more components of the monitoring and treatment device 100. As an example, the device controller 106 may include a touch screen 402 configured to display a visual alarm, such as an icon or text indicating the impending therapeutic shock. As another example, the controller 400 may include a speaker 404 configured to emit an audio alarm. The audio alarm may be, for instance, a bell, a gong, a verbal warning or instruction (e.g., warning the patient 104 and any bystanders of the impending therapeutic shock, instructing the patient 104 to push a response button to delay or cancel the impending therapeutic shock, instructing bystanders to stand back from the patient 104, and/or so on). As another example, as noted above, the signal processing unit 310 may include a tactile device configured to vibrate to provide a haptic alarm. The one or more alarms may, in implementations, be combinations of visual, audio, and/or haptic alarms that may escalate over time. For instance, an audio alarm may become louder over a predetermined alarm period.

If the patient 104 is still conscious when the controller 400 initiates one or more alarms, the patient 104 may be able to push one or more response buttons to signal to the controller 400 that the one or more therapeutic pulses should be delayed or cancelled. For example, the controller 400 may include response buttons 406 provided on opposite sides of the controller 400 that the patient 104 must push simultaneously to delay or cancel the therapeutic shock. In implementations, the wearable cardiac device may include a separate response button unit that contains the one or more response buttons, similar to the patient interface pod 110 shown in FIG. 1. As an illustration, the electrode belt 300 may include a wired response button unit (e.g., a response button unit incorporated as an integrated part of the electrode belt 300 or a response button unit that connects to the signal processing unit 310). The response button unit may be configured to removably connect to the cardioprotective apparel 200 (e.g., similar to the patient interface pod 110 attached to the cardioprotective apparel 101 in FIG. 1, via a clip, snaps, buckles, etc.) or to clothes of the patient (e.g., via a clip). As another illustration, the monitoring and treatment device 100 may include a wireless response button unit, for example, implemented as a watch or wristband that the patient wears along with the cardioprotective apparel 200. As another illustration, a patient user device, such as a smart phone, may serve as the response button unit. For example, the controller 400 may communicate with the smart phone (e.g., via Bluetooth®, via cellular networks, etc.) and cause the smart phone to display a button, a link, or so on that the patient 104 must press to delay or cancel the therapeutic shock. In implementations, the monitoring and treatment device 100 may include combinations of the above types of response buttons.

In implementations, the controller 400 may also send outputs and receive inputs from the patient 104 or other user unrelated to an impending therapeutic shock. To illustrate, the controller 400 may display a settings menu to a user via the touch screen 402 that the user (e.g., a caregiver for the patient 104, such as a doctor or device technician) can use to input settings for the monitoring and treatment device 100. Examples of settings may include energy levels for the one or more therapeutic shocks, the timing between therapeutic shocks, the length of the predetermined alarm period, and/or the like.

As noted above, the electrode belt 300 includes one or more components configured to be assembled into the cardioprotective apparel 200 before the monitoring and treatment device 100 is worn and used by the patient 104 to protect the patient 104 from treatable cardiac arrhythmias. For example, in implementations and as shown in FIG. 2, the cardioprotective apparel 200 may include pockets 208 for receiving the therapeutic electrodes 304 of the electrode belt 300, which may be secured (after the therapeutic electrodes 304 have been inserted into the pockets 208) by pocket snaps 210 (e.g., provided on the underside of the pocket fabric, as illustrated by the dotted lines in FIG. 2). The pockets 208 may include multiple sets of pockets, such as pockets 208a provided on the portion of the cardioprotective apparel 200 configured to lie against the patient's back and a pocket 208b provided on the band 204. In implementations, the cardioprotective apparel 200 may include electrode fasteners 212 configured to receive the sensing electrodes 302 of the electrode belt 300. In implementations, the cardioprotective apparel 200 may also include an enclosure 214 for the signal processing unit 310 of the electrode belt 300. As shown in FIG. 2, the enclosure 214 may include a fabric flap 216 configured to be folded over the signal processing unit 310 and secured in place via flap snaps 218. Alternatively, or additionally, the location of the enclosure 214 shown in FIG. 2 may include a housing that the signal processing unit 310 is configured to removably snap or otherwise attach into.

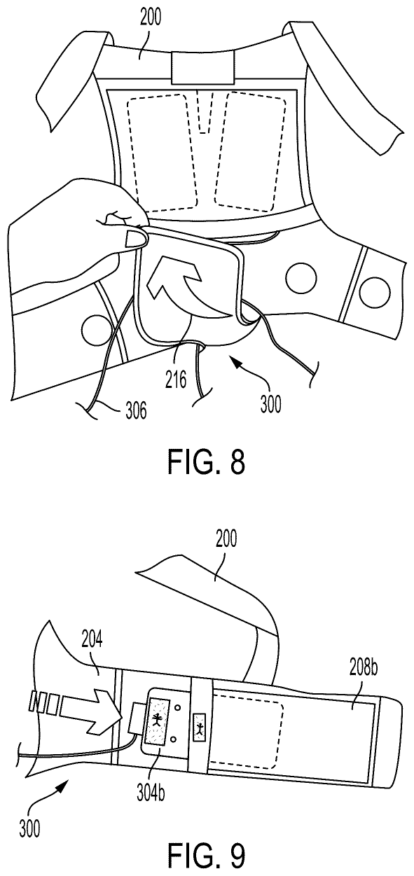

An example assembly process for a monitoring and treatment device 100 including the cardioprotective apparel 200 and the electrode belt 300 is illustrated in FIGS. 5-10. To assemble the electrode belt 300 into the cardioprotective apparel 200, first, the patient 104 or other user inserts therapeutic electrodes 304a into the pockets 208a provided on the portion of the cardioprotective apparel 200 configured to contact the patient's back, as shown in FIG. 5. Once the therapeutic electrodes 304a are inserted into the pockets 208a, the patient 104 or other user may secure them in place by snapping the pocket snaps 210 of the pockets 208a closed. The patient 104 or other user then places the signal processing unit 310 in the enclosure 214 as illustrated in FIG. 6. In some implementations, placing the signal processing unit 310 in the enclosure 214 may include placing the signal processing unit 310 on the fabric flap 216 at the location shown in FIG. 6. In other implementations, placing the signal processing unit 310 in the enclosure 214 may include snapping the signal processing unit 310 into a housing provided at the enclosure 214 location shown in FIG. 6. In implementations, correctly placing the signal processing unit 310 in the enclosure 214 may include arranging the wires 306 of the electrode belt 300 within the enclosure 214, such as according to the arrangement 220 shown in FIG. 7. For example, the patient 104 or other user may place the wires to run between the flap snaps 218 of the enclosure 214, as illustrated in FIG. 7. Once the signal processing unit 310 is placed in the enclosure 214, the patient 104 or other user may close the fabric flap 216 over the signal processing unit 310, as shown in FIG. 8, and secure the fabric flap 216 in place through the flap snaps 218. As shown in FIG. 9, the patient 104 or other user also inserts the final anterior therapeutic electrode 304b into the pocket 208b provided on the band 204 of the cardioprotective apparel 200, securing the therapeutic electrode 304b in place by snapping closed the pocket snaps of the pocket 208b (not shown but provided on the underside of the pocket 208b, similar to pocket snaps 210 described above).

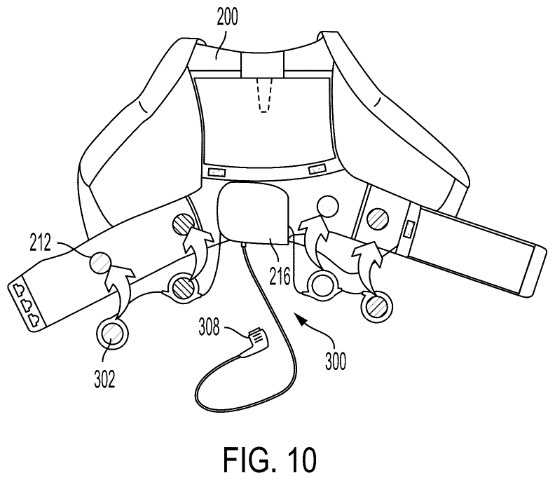

Finally, as illustrated in FIG. 10, to finish the assembly of the electrode belt 300 within the cardioprotective apparel 200, the patient 104 or other user places the sensing electrodes 302 on the electrode fasteners 212 of the cardioprotective apparel 200. In implementations, the sensing electrodes 302 may be removably attached to the electrode fasteners 212 using any of a variety of removable attachment mechanisms. For example, a back side opposite to a sensing surface of each sensing electrode 302 may include hook or loop fabric configured to be adhered to loop or hook fabric of a corresponding electrode fastener 212. As another example, each electrode fastener 212 may include a pocket (e.g., with a conductive surface) configured to receive a sensing electrode 302. As another example, each electrode fastener 212 may include a housing that a sensing electrode 302 may be popped into, screwed into, etc. In implementations, each of the sensing electrodes 302 may be configured to be placed onto a specific corresponding electrode fastener 212 as shown in FIG. 10. To illustrate to the patient 104 or other user which sensing electrode 302 should be placed on which electrode fastener 212, each sensing electrode 302 may be provided with a color, a shape, a size, a symbol, and/or the like that matches a corresponding color, shape, size, symbol, and/or the like on the correct electrode fastener 212 the sensing electrode 302 should be affixed to.

Once the electrode belt 300 is correctly assembled into the cardioprotective apparel 200, the connector 308 may be plugged into the controller 400, and the patient 104 may put on the cardioprotective apparel 200. For example, the assembly shown in FIGS. 5-10 is performed with the assemblable elements of the electrode belt 300 being inserted onto the inner surface of the cardioprotective apparel 200 configured to be placed against the patient's skin when worn. As such, once the assembly is finished, the patient 104 may put on the assembled cardioprotective apparel 200 with the inner surface shown in FIGS. 5-10 against their skin, securing the cardioprotective apparel 200 in place using the clasp 206. FIGS. 5-10 show an example of how the electrode belt 300 may be assembled into the cardioprotective apparel 200, but other assembly steps or methods may be used. To illustrate, as discussed above, the electrode belt 300 may not include sensing electrodes 302 in some implementations. Instead, the sensing electrodes may be an integrated part of the cardioprotective apparel 200. In such implementations, the assembly of the electrode belt 300 into the cardioprotective apparel 200 may not include the step shown in FIG. 10. As another illustration, while the assembly of the electrode belt 300 into the cardioprotective apparel 200 shown in FIGS. 5-10 is done with the assemblable components of the electrode belt 300 being inserted or attached to the inner surface of the cardioprotective apparel 200 configured to be placed against the patient's skin, in some implementations, the assemblable components of the electrode belt 300 may be inserted or attached to the outer surface of the cardioprotective apparel 200 configured to face away from the patient's skin when worn. For instance, the pockets 208 may be additionally or alternatively accessible from the outer surface of the cardioprotective apparel 200, the enclosure 214 may be provided on the outer surface, and/or the electrode fasteners 212 may include an aperture whereby the sensing surface of the sensing electrodes 302 may contact the patient's skin surface when the sensing electrodes 302 are attached to the electrode fasteners 212 via the outer surface of the cardioprotective apparel 200.

However, in various implementations, the result of the assembly of the monitoring and treatment device 100 is that the cardioactive band 204 supports at least one anterior therapeutic electrode and a number of sensing electrodes distributed in a spaced apart configuration. For instance, FIG. 11 shows an illustration of the monitoring and treatment device 100 of FIGS. 2-10 being worn by the patient 104. As illustrated in FIG. 11, the cardioactive band 204 supports at least one anterior therapy electrode (e.g., therapeutic electrode 304b) and ECG sensing electrodes (e.g., sensing electrodes 302) in a spaced apart configuration such that the at least one anterior therapy electrode 304b and the sensing electrodes 302 are disposed again predetermined anatomical regions on the patient 104 when the monitoring and treatment device 100 is worn by the patient 104. More specifically, the at least one anterior therapy electrode 304b may be disposed on an anterior electrode placement region and the sensing electrodes 302 may be disposed on predetermined ECG sensing anatomical regions when the monitoring and treatment device 100 is worn by the patient 104. As an illustration, FIG. 11 shows an example predetermined ECG sensing anatomical region 500 for a sensing electrode 302 and an example anterior therapy electrode placement region 502 for an anterior therapy electrode 304b.

As described above, the at least one anterior therapy electrode and the ECG sensing electrodes need to remain in their respective anatomical regions on the patient 104 when the patient 104 is wearing the monitoring and treatment device 100. However, patient anatomy can displace the at least one anterior therapy electrode and/or at least one of the ECG sensing electrodes. For instance, the patient's breast tissue can make it difficult for the at least one therapy electrode and/or the anterior ECG sensing electrodes to sit flush with the patient's skin. As another example, front torso projections of the patient 104 can similarly make it difficult for the at least one therapy electrode and/or the anterior ECG sensing electrodes to sit flush with the patient's skin, as well as potentially make the cardioprotective apparel of the monitoring and treatment device 100 more uncomfortable for the patient 104 to wear. Accordingly, the monitoring and treatment device 100 may include one or more features configured to better accommodate patient anatomy across different sizes to better facilitate the electrodes in lying flat against or near the patient's skin.

In implementations, the at least two shoulder harnesses of the monitoring and treatment device 100 are configured to shift breast tissue of the upper torso region of the patient 104 away from predetermined regions for the electrodes of the monitoring and treatment device 100. For example, each of the at least two shoulder harnesses may be configured to shift the patient's breast tissue away from the anterior therapy electrode placement region (e.g., region 502) on the patient 104 and/or at least one of the predetermined ECG sensing anatomical regions (e.g., region 500) on the patient 104. The shifting of the breast tissue of the upper torso region of the patient 104 is configured to facilitate the at least one therapy electrode and the plurality of ECG sensing electrodes in lying in a flush manner with the patient's skin when the monitoring and treatment device 100 is worn by the patient 104.

In implementations, each of the shoulder harnesses of the monitoring and treatment device 100 may include a force applicator configured to shift the breast tissue of the upper torso region of the patient 104. The force applicator is configured to apply a force to the patient's breast tissue to facilitate the shifting as described above. For instance, the force applicator may include an elastic surface configured to apply force or pressure within a predetermined range to shift the patient's breast tissue, such as a pressure of no more than 0.65 psi. In examples, the force applicator may include a fabric piece configured to shift the breast tissue of the upper torso region of the patient 104, such as based on a predetermined elasticity due to the materials and/or construction of the fabric piece. Such a fabric piece may be a sling portion, as described in further detail below. In examples, the force applicator may be the shoulder harness itself. The shoulder harness may be configured in a cut or pattern predetermined to cause the shifting of the breast tissue and/or be constructed of materials (e.g., having a predetermined elasticity) configured to cause the shifting of the breast tissue. The cut, pattern, construction, material, and/or elasticity (e.g., the material elasticity of the fabric forming the shoulder harness) of the shoulder harnesses may thus form the force applicator in such implementations.

An example of how the at least two shoulder harnesses can shift and/or shape the patient's breast tissue away from predetermined electrode regions on the patient 104 is shown in FIG. 12. FIG. 12 illustrates an example of the monitoring and treatment device 100 that includes cardioprotective apparel 600 having shoulder harnesses 602 attached to a cardioactive band 604 configured to be disposed on an inferior end of the upper torso region of the patient 104. Each of the shoulder harnesses 602 also includes a sling portion 606 positioned at the juncture of the shoulder harness and the cardioactive band 604. The sling portion 606 is configured to shift the patient's breast tissue away from predetermined anatomical regions on the patient 104, such as an anterior therapy electrode placement region for at least one anterior therapy electrode 608 and/or and predetermined ECG sensing anatomical regions for at least one ECG electrode 610. For example, the shoulder harnesses 602 are configured to be disposed along lateral ends of the upper torso region of the patient 100 when the monitoring and treatment device 100 is worn by the patient 104 (e.g., disposed at lateral portions of the patient's shoulders) such that each sling portion 606 is configured to contact a lateral portion of the patient's breast tissue, as shown. Additionally, the sling portion 606 may serve to minimize harsh edges around the breast tissue where the shoulder harnesses 602 lie against the breast tissue. In implementations, the sling portion 606 may further help to stabilize breast tissue within a bra worn over the monitoring and treatment device 100, as described above.

The sling portion 606 may be formed of a fabric or a composite of fabrics having an elasticity configured to shift the patient's breast tissue, as described above. In implementations, the fabric of the sling portion 606 may also be configured with enough range of elasticity to accommodate different volumes of breast tissue. In examples, the fabric of the sling portion 606 may be a mesh, a knit, a stretch woven, and/or combinations of such materials. In examples, the fabric of the sling portion 606 may include a number of layers bonded together (e.g., with an adhesive) to achieve the desired amount of tissue displacement.

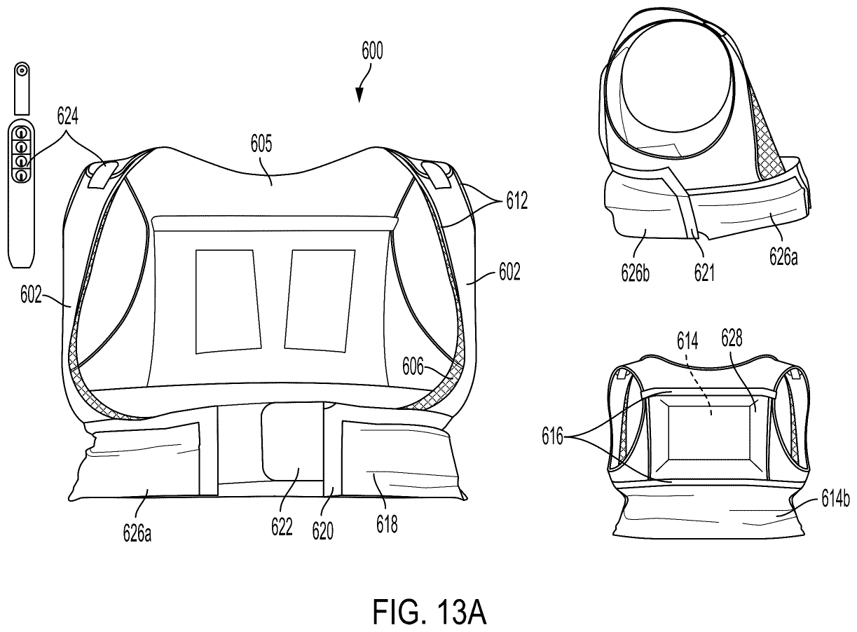

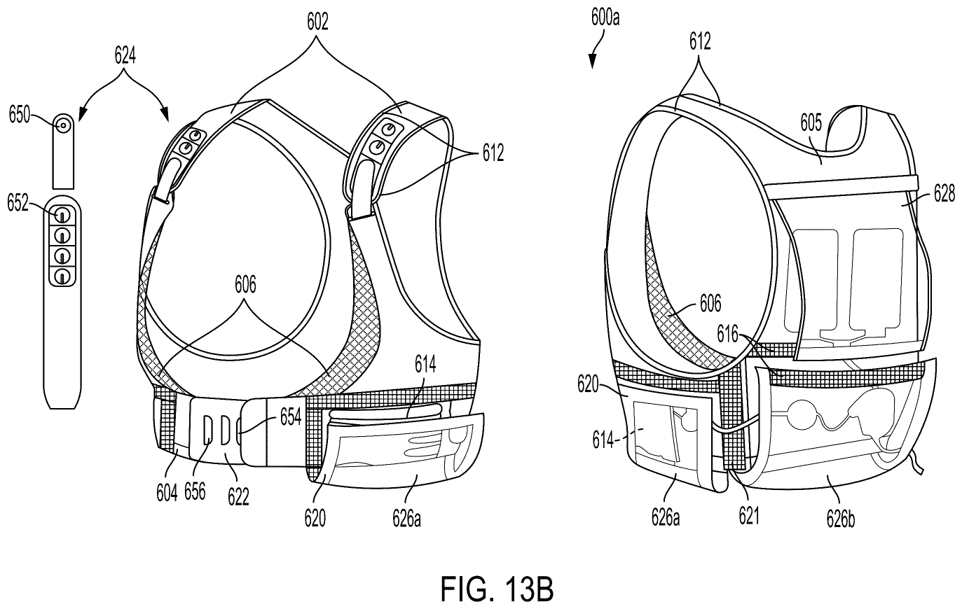

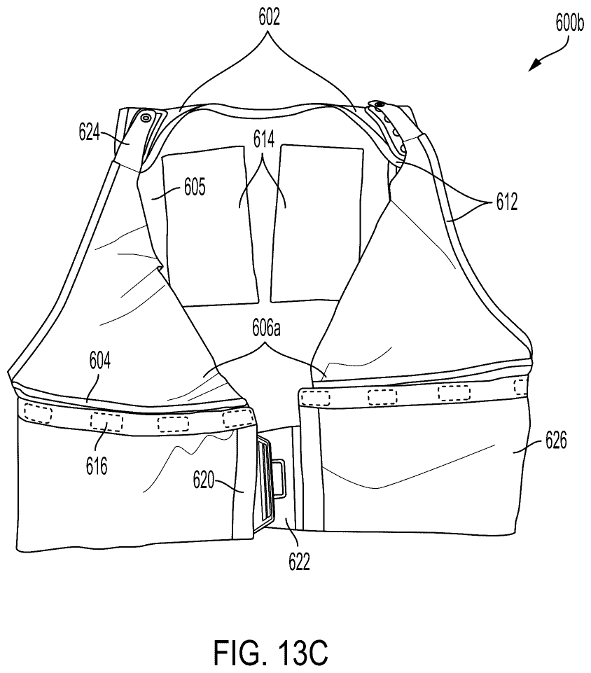

FIG. 13A shows additional views of the cardioprotective apparel 600 from FIG. 12. In implementations, the cardioprotective apparel 600 may include further features to improve the functionality and comfort of the monitoring and treatment device 100. For example, the cardioprotective apparel 600 may include smooth binding or elastic 612 around armholes and the neckline for patient comfort. Additionally, the shoulder harnesses 602 may be configured with wide shoulder bands with a low profile that, as shown in FIG. 12, are worn along lateral ends of the patient's upper torso region (e.g., lateral ends of the patient's shoulders). This fitting of the shoulder harnesses 602 may provide for patient comfort by minimizing pressure points on the shoulder harnesses 602 through the wide, low shoulder bands, Moreover, for example and returning to FIG. 13A, the placement of adjustment mechanisms 624 on the shoulder harnesses 602 may help to minimize overlap between a bra worn by the patient 104 and the shoulder harnesses 602. The adjustment mechanisms 624 for the shoulder harnesses 602 may adjust the length of the shoulder harnesses 602 while simultaneously adjusting the shape of the sling portions 602 and the shoulder harnesses 602. As shown, the adjustment mechanisms 624 may be relatively flat and disposed on the front of the shoulder harnesses 602 to avoid interfering with and/or overlap with strap adjustment mechanisms on a bra worn by the patient 104, which may be commonly bulkier and disposed on the back of the patient 104.

In examples, a clasp or closure 622 for the cardioprotective apparel 600 may be provided on the cardioactive band 604. The closure 622 may similarly be low-profile, wide, and adjustable with cushioning to protect the patient 104. In examples, the closure 622 and the adjustment mechanisms 624 may be configured similarly, such as with the same type or similar type of easy open, easy close snaps, hooks, shells, buckles, slide connectors, etc. In examples, the closures 622 and the adjustment mechanisms 624 may be different. For instance, the closure 622 may completely separate to allow the patient 104 to put on and take off the monitoring and treatment device 100, and the adjustment mechanisms 624 may be configured to only allow lengthening or shortening of the shoulder harnesses 606. The closure 622 and the adjustment mechanisms 624 may be configured for easy operation for a range of patients 104, including patients with motor skill issues and mobility issues. Examples of the adjustment mechanism 624 and the closure 622 are described below with respect to FIG. 13B. In implementations, and as shown in FIG. 13A, the bottom edge of the closure 622 may be offset from the bottom edge of the fabric forming the cardioactive band 604 around the closure 622. Offsetting the closure 622 from the bottom edge of the cardioactive band 604 may better accommodate, for instance, front torso projections and help keep the closure 622 from digging into the skin of the patient.

With respect to assembly of the monitoring and treatment device 100 including the cardioprotective apparel 600, exterior pockets or receptacles 614 for the components of the electrode belt 300 may be provided on an exterior of the cardioprotective apparel 600 (e.g., on the side of the cardioprotective apparel 600 facing away from the patient's skin). For instance, in implementations, the receptacles 614 may be configured to receive the sensing electrodes, therapy electrodes, a signal processing unit, etc. of the electrode belt 300. In implementations, the receptacles 614 may include at least one anterior pocket disposed on an anterior portion of the cardioactive band 604 and configured to receive at least one anterior therapy electrode (e.g., electrode 304b) and at least one posterior pocket disposed on a back surface 605 of the cardioprotective apparel 600 and configured to receive at least one posterior therapy electrode (e.g., electrode 304a). In this sense, the receptacles 614 may be configured similarly to the electrode fasteners 212, pockets 208, and/or enclosure 214 shown and described above with reference to FIGS. 2 and 5-10. With respect to the exterior receptacles 614, the cardioprotective apparel 600 includes an inner side configured to face the patient's skin when worn and an outer side configured to face away from the patient's skin when worn. As such, the exterior receptacles 614 (e.g., including anterior receptacle(s) for anterior therapy electrode(s), posterior receptacle(s) for posterior therapy electrode(s), receptacles for sensing electrodes, and/or a receptacle for a signal processing unit) may be accessible from the outer side of the cardioprotective apparel 600.

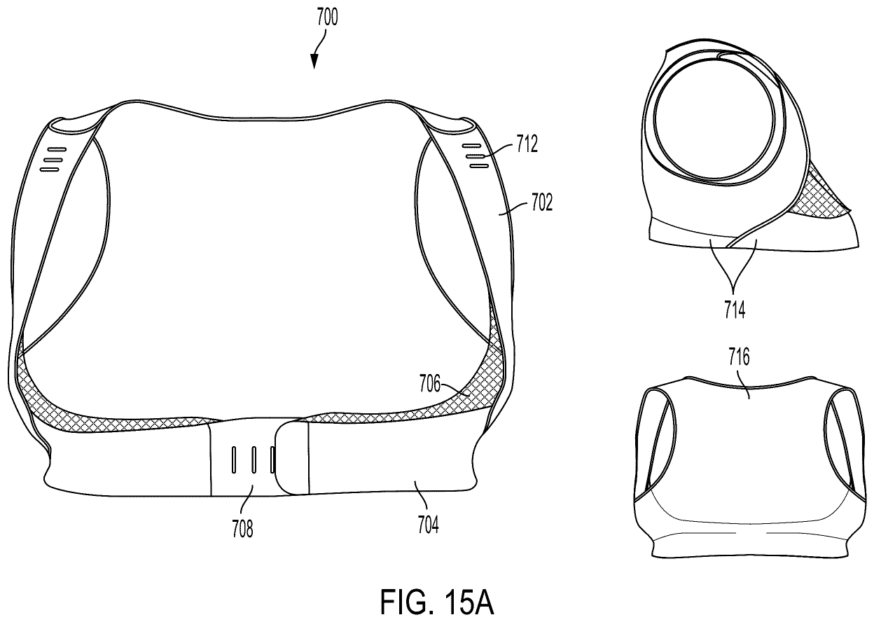

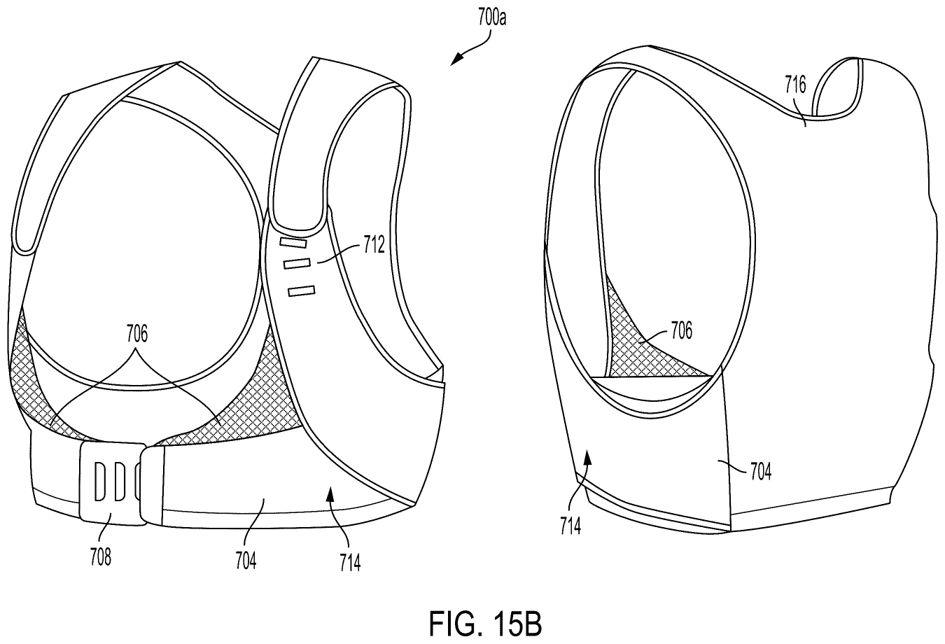

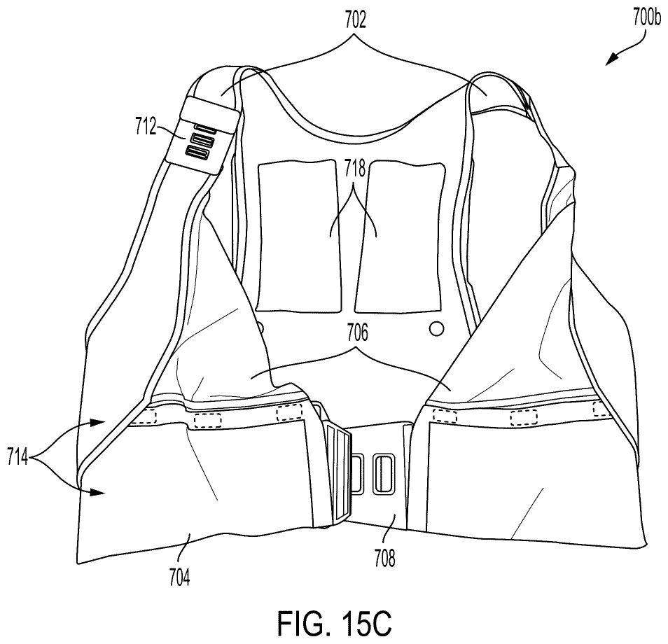

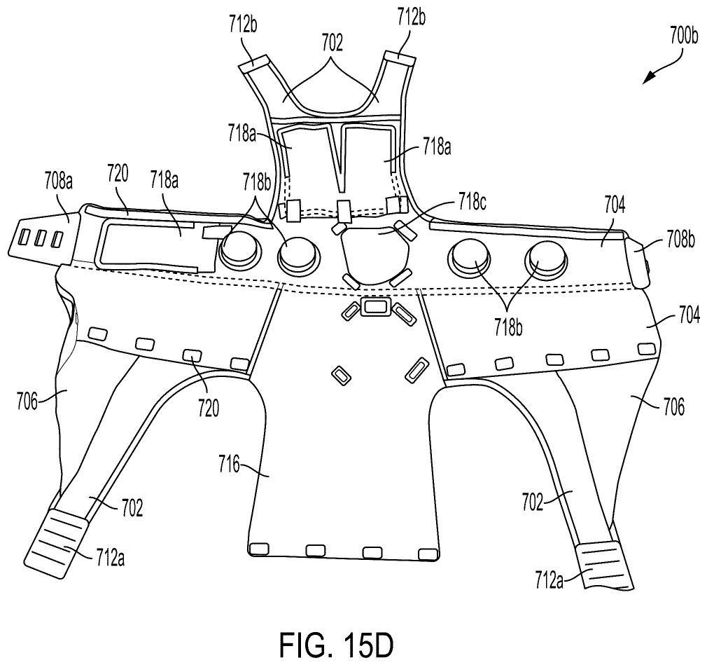

The exterior receptacles 614 may be bonded to the cardioactive band 604 and the posterior or back surface 605 of the cardioprotective apparel 600 and keep, for instance, cords or cables of an electrode belt from directly being pressed against the patient's skin. In addition, the exterior receptacles 614 may be covered by exterior flaps configured to hold the components of the electrode belt 300 in place and prevent the cords from catching once assembled. For example, the exterior flaps may include band flaps 626a and 626b configured to cover the receptacles 614 and other components of the electrode belt 300 assembled onto the cardioactive band 604 and a back flap 628 configured to cover the receptacles 614 and other components of the electrode belt 300 on the back surface 605 of the cardioprotective apparel 600. For instance, the band flap 626a may be configured to cover the receptacle 614 for the anterior therapy electrode 304b, the band flap 626b may be configured to cover the signal processing unit 310, and the back flap 628 may be configured to cover the receptacles 614 for the posterior therapy electrodes 304a. The band flaps 626a, 626b may also be configured to cover receptacles 614 configured to receive sensing electrodes. Other implementations of the exterior flaps are shown, for example, in FIG. 13B shown and described below. Alternatively, in implementations, the cardioprotective apparel 600 may include fewer or additional exterior flaps. For example, the cardioprotective apparel 600 may include one exterior flap similar to the cardioprotective apparel 700b shown and described below with reference to FIGS. 15D and 15D.

The exterior flaps, including the band flaps 626a, 626b and the back flap 628, may further include detachable hook-and-loop fabric fasteners or other types of easily detachable fasteners 616, such as snaps or buttons, for interlocking the exterior flaps in place. As an example, the back flap 628 may include both hook-and-loop fasteners and a snap in the center to secure the back flap 628 in place. The exterior flaps may also be implemented on the cardioprotective apparel 600 to maximize the wearability of the cardioprotective apparel 600. For instance, the band flaps 626a and 626b may also overlap at back side seam areas 621 for better stretch and to avoid exposure of cables (e.g., as part of the electrode belt 300).

In implementations, at least portions of the exterior flaps may be shaped to better cover and hold in place the assembled components of the electrode belt 300. As an illustration and as shown in FIG. 13A, the band flap 626a may include pre-gathering 618 to help better accommodate and secure, for example, the anterior therapy electrode within an anterior therapy electrode receptacle 614. In examples, the back flap 628 may include similar pre-gathering for receptacles 614 configured to receive the posterior therapy electrodes. In examples, the exterior flaps may include pre-gathering or other shaping for receiving other components of the electrode belt 300, such as the band flap 626b having pre-gathering or other shaping configured to surround the signal processing unit 310. Interaction points 620 for the cardioprotective apparel 600 (e.g., points where the patient 104 interacts with the cardioprotective apparel 600 to assemble the monitoring and treatment device 100) may be bonded and draw the patient's eye visually, as shown in FIG. 13A.

As such, the above-discussed features of the cardioprotective apparel 600 may help the components of the electrode belt 300 remain in place against desired anatomical locations and resist displacing forces. For example, compression forces exerted by the exterior flap 626a, the pre-gathering 618 around the receptacle 614, the shape of the clasp 622, and the shifting of the patient's breast tissue by the tissue slings 606 may configure the anterior therapy receptacle 614 to resist transverse rotational forces exerted by underlying tissue of the patient's upper torso region (e.g., based on front torso projections). In turn, this may help the anterior therapy electrode (e.g., electrode 304b) lie in a flush manner with the patient's skin when the monitoring and treatment device 100 is worn by the patient 104. Similar features may provide similar benefits to the sensing electrodes of the electrode belt 300 configured to contact and anterior portion of the patient's upper torso. Additionally, like benefits may be conferred by other features of the cardioprotective apparel 600a and 600b discussed below.

FIG. 13B illustrates views of another implementation of the cardioprotective apparel 600, referred to in FIG. 13B as cardioprotective apparel 600a. The cardioprotective apparel 600a is generally similar to the cardioprotective apparel 600 but with exterior flaps that are in a different configuration. FIG. 13B also highlights the exterior flaps including the band flaps 626a, 626b on the cardioactive band 604 and the back flap 628 on the back of the cardioprotective apparel 600. As shown in FIG. 13B, the electrode belt 300 can be assembled into the cardioprotective apparel 600a by opening the exterior flaps and inserting the components of the electrode belt 300 into corresponding receptacles 614. For example, the anterior therapy electrode 304b can be inserted into the anterior therapy electrode receptacle 614 on the cardioactive band 604 under the band flap 626a, as illustrated. Once the electrode belt 300 is assembled into the cardioprotective apparel 600a, the exterior flaps can be closed using the fasteners 616. As shown, the fasteners 616 also overlap on the band flap 626b such that the band flap 626b is closed using a fastener 616 provided on the inside surface of the band flap 626b that mates with a fastener 616 on the back surface 605. The back flap 628 is closed using a fastener 616 that mates with a fastener 616 provided on the outside surface of the band flap 626b. The method of assembly shown in FIG. 13B may be beneficial, for example, because it keeps the cables of the electrode belt 300 off of the body of the patient 104, providing for additional comfort, while also covering the cables of the electrode belt 300 to prevent the cables from catching while the patient 104 is wearing the monitoring and treatment device 100.

Additionally, FIG. 13B illustrates examples of the adjustment mechanisms 624 and the closure 622. With respect to the adjustment mechanism 624, in the example shown in FIG. 13B, the adjustment mechanism 624 may include slidable snaps, where a portion of shoulder harness 602 includes a cap 650 (e.g., the male portion of the slidable snap) and the other portion of the shoulder harness 602 includes a number of sockets 652 (e.g., the female portion of the slidable snap), each with a groove configured to receive the cap. Using the groove, the patient 104 can thus slide the cap 650 into the corresponding socket 652 that provides for the best fit of the cardioprotective apparel 600 for the patient 104. To readjust the shoulder harness 602, the patient 104 can slide the cap 650 out of the first socket 652 and repeat the process with a second socket 652. With respect to the closure 622, in the example shown in FIG. 13B, the closure 622 may include a tab 654 on one portion of the cardioactive band 604 and a series of grooves 656 configured to receive the tab 654 on the other portion of the cardioactive band 604. The patient 104 can close the closure 622 by inserting the tab 654 into the corresponding groove 656 that provides for the best fit of the cardioprotective apparel 600 for the patient 104. The patient 104 inserts the tab 654 into the groove 656 at an angle and then flattens the tab 654 to lock it in place within the groove 656. To open the closure 622, the patient 104 can remove the tab 654 from the groove 656 by pulling the tab 654 out at an angle.

FIG. 13C illustrates a view of another implementation of the cardioprotective apparel 600, referred to in FIG. 13C as cardioprotective apparel 600b. The cardioprotective apparel 600b is generally similar to the cardioprotective apparel 600. However, the cardioprotective apparel 600b includes force applicators 606a configured as each of the shoulder harnesses 602, rather than force applicators configured as a sling portion as discussed above. These force applicators 606a may similarly apply a force to the patient's breast tissue to shift the breast tissue away from, for example, electrode placement regions on the patient 104. With the force applicators 606a, the force is tailored using the cut and materials of the shoulder harnesses 602, instead of by including a separate sling portion. Regardless, the force applicators 606a similarly serve the goal, not of supporting the patient's breast tissue, but moving the breast tissue away from the electrode placement regions. In addition to the different implementation of the force applicators 606a, the cardioprotective apparel 600b includes a single band flap 626 that extends around the circumference of the cardioprotective apparel 600b, rather than the multiple band flaps discussed above. Further, in implementations, the cardioprotective apparel 600b does not include separate band and back flaps but includes a single full-length back panel, similar to the cardioprotective apparel 700 discussed below. As shown, in implementations, the cardioprotective apparel 600 may include a front closure 622 in a tapered or conical configuration with multiple adjustment settings (e.g., with adjustment settings similar to the closure 622 shown in FIG. 13B). The tapered front closure 622 may better accommodate front torso protrusions than a non-tapered closure, as well as be more comfortable for the patient 104 to wear for extended periods of time by minimizing pinching and/or pressure zones at the closure 622.

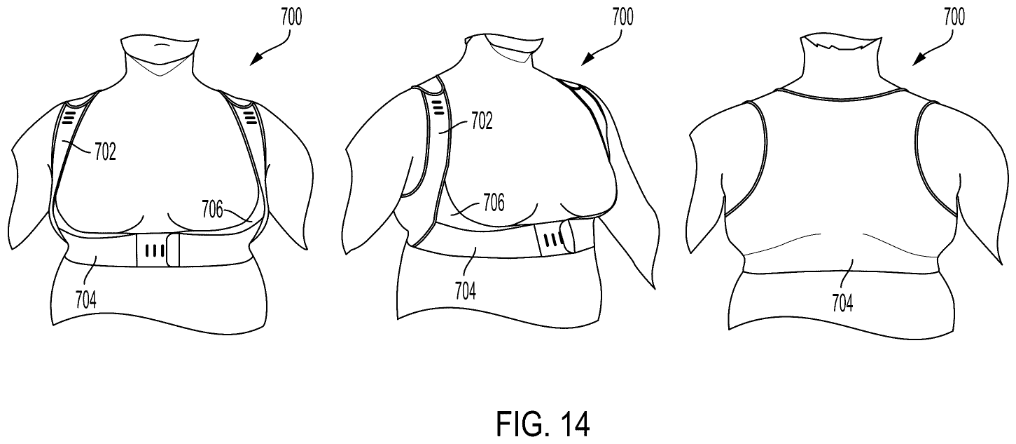

Another example of how the at least two shoulder harnesses can shift and/or shape the patient's breast tissue away from predetermined electrode regions on the patient 104 is shown in FIG. 14. In FIG. 14, the monitoring and treatment device 100 includes cardioprotective apparel 700 having shoulder harnesses 702 attached to a cardioactive band 704 configured to be disposed on an inferior end of the upper torso region of the patient 104. Similar to the example shown in FIG. 12, the monitoring and treatment device 100 illustrated in FIG. 14 includes a sling portion 706 at each of the shoulder harnesses 702. More specifically, the sling portion 706 is located where the shoulder harness 702 meets the cardioactive band 704. Also similarly to the example shown in FIG. 12, the sling portions 706 are configured to shift the patient's breast tissue away from predetermined anatomical regions on the patient 104, such as predetermined anatomical regions associated with one or more electrodes. For example, in implementations, the sling portions 706 may be made of a power mesh configured to shift the breast tissue away from the top of the cardioactive band 704.

FIG. 15A shows additional views of the cardioprotective apparel 700 from FIG. 14. In implementations, the cardioprotective apparel 700 may include further features to improve the functionality and comfort of the monitoring and treatment device 100. These features may be the same as similar features for the cardioprotective apparel 600 described above with reference to FIGS. 13A and 13B and/or these features may be different than those described above with reference to FIGS. 13A and 13B. The shoulder harnesses 710 of the cardioprotective apparel 700 may be wide and padded for additional comfort for all-day wear. An adjustment mechanism 712 for the shoulder harnesses 710 may be at the front armhole to adjust the length of the shoulder harnesses 710. In implementations, the cardioprotective apparel 700 may also include a full-length back panel 716. As shown in FIG. 15A, the full-length back panel 716 may be provided on an exterior side of the cardioprotective apparel 700 (e.g., on a side facing away from the patient 104 when the patient 104 is wearing the monitoring and treatment device 100). In implementations, the full-length back panel 716 may perform a function similar to the exterior flaps shown and described with reference to the cardioprotective apparel 600. As such, the full-length back panel 716 may cover receptacles configured to receive components of the electrode belt 300 through an exterior assembly process, as well as components of the electrode belt 300 once assembled into the cardioprotective apparel 700. Thus, in implementations, using the adjustment mechanisms 712 to shorten the shoulder harnesses 702 may provide additional compression adjustment across the back panel 716 of the cardioprotective apparel 700.

As with the cardioprotective apparel 600, 600a, and 600b discussed above, the adjustment mechanisms 712 and a clasp or closure 708 of the cardioprotective apparel 700 may be configured as easy to open and close. For example, the adjustment mechanisms 712 and the closure 708 may be configured as similarly with tabs and a series of grooves to provide fit adjustment for the cardioprotective apparel 700. The cardioprotective apparel 700 may also include a spacer fabric 714 throughout to provide comfort, breathability, and support (e.g., particularly since the cardioprotective apparel 700 may include multiple layers due to the full-length back panel 716) while remaining lightweight.

FIG. 15B illustrates views of another implementation of the cardioprotective apparel 700, referred to in FIG. 15B as cardioprotective apparel 700a. As shown in FIG. 15B, in implementations, the adjustment mechanism 712 may be provided lower down on the shoulder harness 702, such as about halfway down the shoulder harness 702. Positioning the adjustment mechanism 712 down on the shoulder harness 702, for example, may further reduce the layering of bulky sections of a bra over the adjustment mechanism 712 and be easier for the patient 104 to use the adjustment mechanism 712 while wearing the monitoring and treatment device 100 to change the length of the associated shoulder harness 702.

FIGS. 15C and 15D illustrate views of another implementation of the cardioprotective apparel 700, referred to in FIGS. 15C and 15D as cardioprotective apparel 700b. As shown in FIG. 15C, the cardioprotective apparel 700b is generally similar to the cardioprotective apparel 700. However, as shown in FIG. 15C, in implementations the cardioprotective apparel 700b may include a conical or tapered front closure 708, similar to the front closure 622 shown in FIG. 13C. Additionally, FIG. 15C also illustrates receptacles 718 configured to receive therapy electrodes when the electrode belt 300 is assembled into the cardioprotective apparel 700b.

FIG. 15D illustrates a view of the cardioprotective apparel 700b with the full-length back panel 716 opened for assembly. The back panel 716 can be opened by opening detachable fasteners 720 positioned throughout the back panel and the rest of the cardioprotective apparel 700b. Accordingly, the surfaces of the cardioprotective apparel 700b shown in FIG. 15D are configured to contact each other once the electrode belt 300 is assembled therein and the back panel 716 is reattached using the detachable fasteners 720. The cardioprotective apparel 700b is also assembled for wear by connecting the two parts of the adjustment mechanisms 712a and 712b on the shoulder harnesses 702 (e.g., with the adjustment mechanism part 712a including a series of grooves that a tab of adjustment mechanism part 712b is inserted into). The patient 104 then secures the cardioprotective apparel 700b on the patient 104 using the two parts of the closure 708a and 708b (e.g., with the closure part 708a including a series of grooves that a tab of closure part 708b is inserted into).

As shown in FIG. 15D, the cardioprotective apparel 700b also includes a number of exterior receptacles 718 configured to receive components of the electrode belt 300 during assembly of the electrode belt 300 into the cardioprotective apparel 700b. The exterior receptacles 718 may be configured similarly to the receptacles 614 discussed above in that the receptacle 718 are accessible from the outer side of the cardioprotective apparel 700b (via the full-length back panel 716). For example, the cardioprotective apparel 700b may include therapy electrode receptacles 718a (e.g., including posterior therapy electrode receptacles on the back surface of the cardioprotective apparel 700b and an anterior therapy electrode receptacle on the cardioactive band 704) configured to receive therapy electrodes and sensing electrode receptacles 718b configured to receive sensing electrodes. The therapy electrode receptacles 718a and sensing electrode receptacles 718b are configured such that the therapy electrodes and sensing electrodes contact the skin of the patient 104 when the patient 104 is wearing and using the monitoring and treatment device 100. As an illustration, the therapy electrode receptacles 718a may be configured as pockets with a conductive mesh on the underside of the pocket (e.g., shown in the view of the receptacles of FIG. 15C). Thus, the therapy electrodes can contact the patient's skin via the conductive mesh. As another illustration, the sensing electrode receptacles 718b may be configured such that a sensing electrode is inserted into a given electrode receptacle 718b via a groove on a side of the electrode receptacle 718b. The underside of the sensing electrode receptacles 718b (e.g., the side not shown in FIG. 15D) may include an opening such that once placed into the sensing electrode receptacle 718b, a sensing electrode can contact the skin of the patient 104 via the opening. As further shown in FIG. 15D, the cardioprotective apparel 700b may include a signal processing receptacle 718c configured to receive a signal processing unit. The signal processing receptacle 718c may include a pocket formed when the detachable fasteners 720 are closed, as illustrated in FIG. 15D. In implementations, the signal processing receptacle 718c may include a soft or hard shell configured to receive the signal processing unit, such as by snapping the signal processing unit into the signal processing receptacle 718c. Similar to the features discussed above with respect to the cardioprotective apparel 600 (as well as the cardioprotective apparel 600a and 600b), these features of the cardioprotective apparel 700, 700a, and 700b may help the components of the electrode belt 300 remain in place against desired anatomical locations and resist displacing forces, such as transverse rotational forces exerted by underlying tissue of the patient's upper torso region.

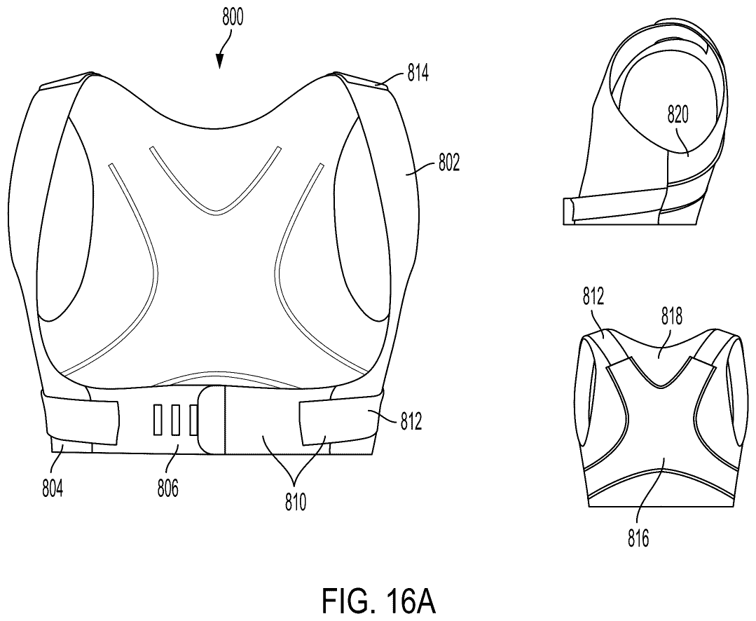

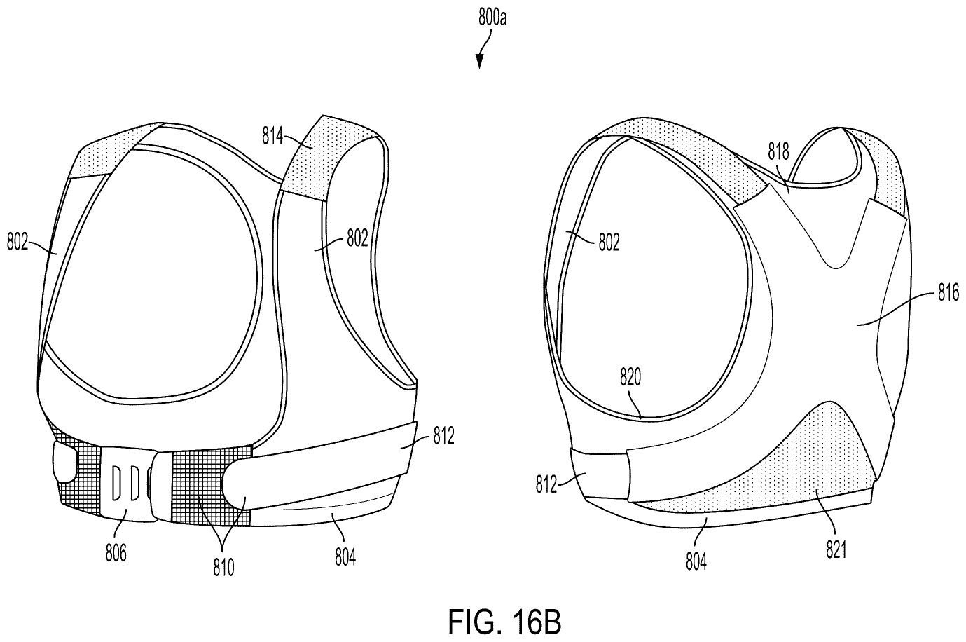

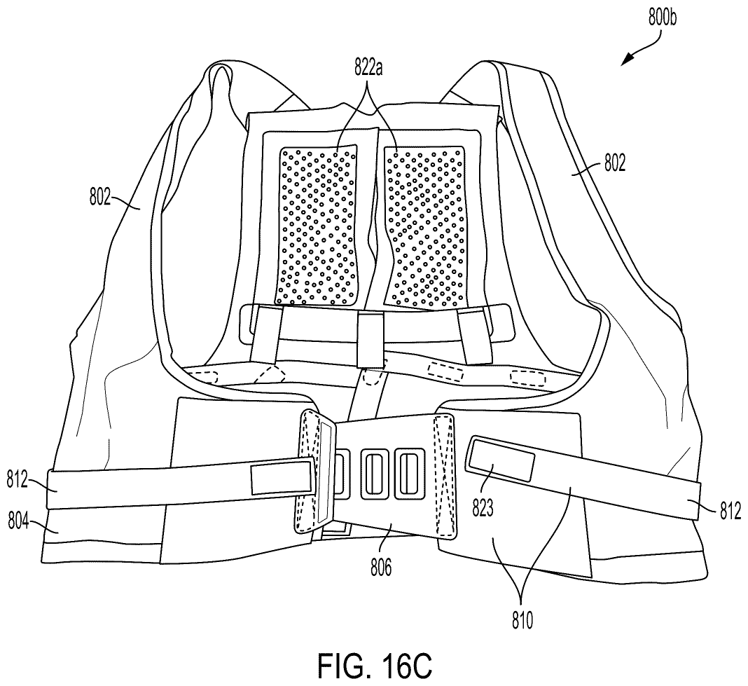

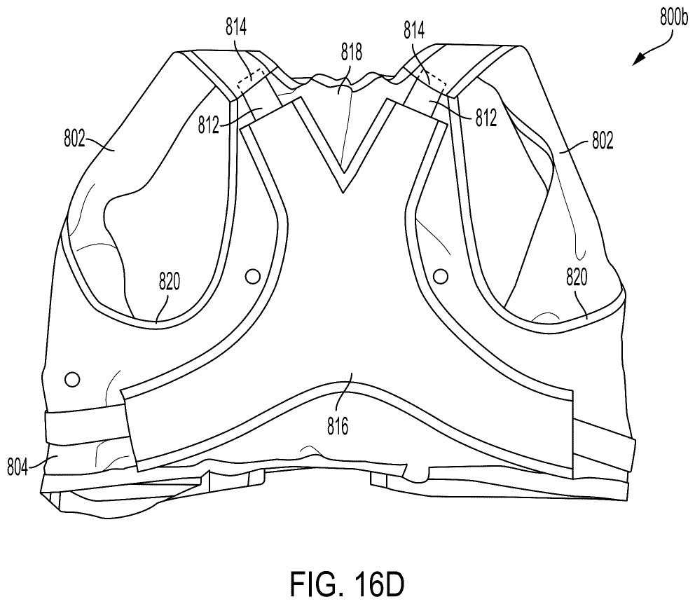

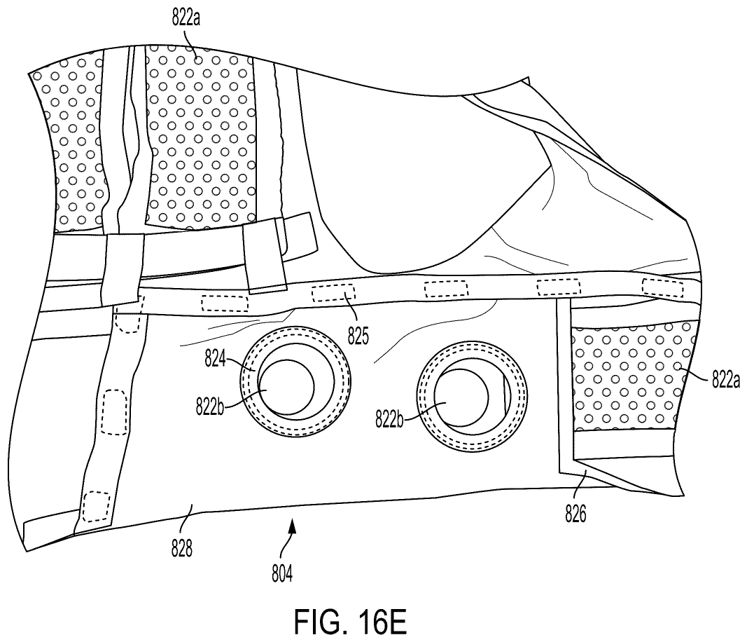

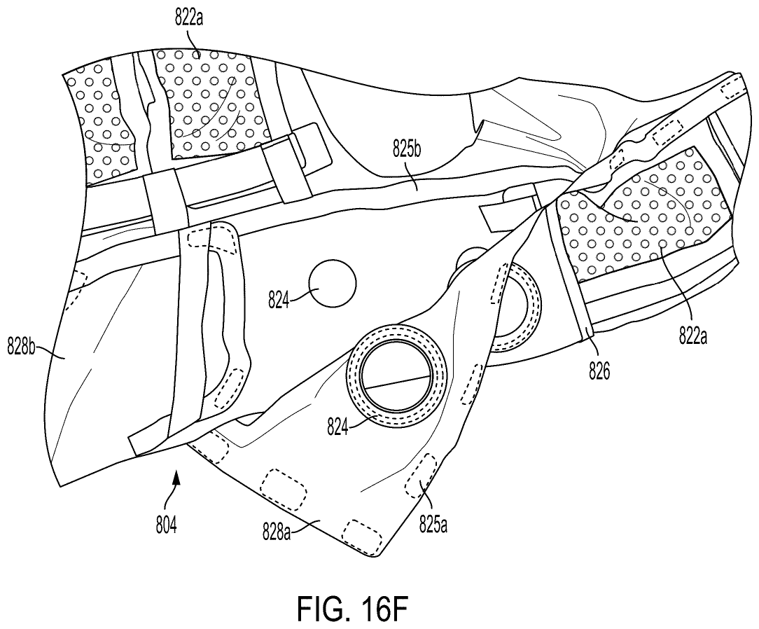

FIG. 16A shows another example of cardioprotective apparel 800 including additional features to improve the functionality and comfort of the monitoring and treatment device 100. As with the cardioprotective apparel discussed above, the cardioprotective apparel 800 includes shoulder harnesses 802 that attach to a cardioactive belt 804. The clasp or closure 806 on the cardioactive belt 804 may be lower profile to accommodate a position under breast tissue. Armholes 820 may also be more closed to allow for a better fit to the patient 104. In implementations, the cut, pattern, construction, material, elasticity, etc. of the shoulder harnesses 802 may serve as force applicators to shift breast tissue of the patient 104, similar to the cardioprotective apparel 600b discussed above. In implementations, the cardioprotective apparel 800 may be assembled from the inside surface (e.g., the surface of the cardioprotective apparel 800 configured to face the patient 104 when worn), an example of which is described below with respect to FIGS. 16C-16F.

In addition, the cardioactive belt 804 may include part of front adjustment mechanisms 810, where the other part of the front adjustment mechanisms 810 are provided on straps 812 that runs from the shoulder harnesses 802 to the cardioactive belt 804. For example, as shown in FIG. 16A and in various implementations, a strap 812 is attached to a given shoulder harness 802 at an attachment point 814 high on the shoulder harness 802. The attachment point 814 being high on the shoulder may, for example, be less likely to interfere with a strap adjustment mechanism on a bra worn by the patient 104. The shoulder harnesses 808 may also be wide to accommodate the added layer of a bra being worn over the shoulder harness 808 and minimize pressure points on the patient's shoulders, as discussed with respect to other embodiments of the cardioprotective apparel above. The strap 812 may run from the attachment point 814 through a knit tunnel 816 on a posterior or back surface 818 of the cardioprotective apparel 800. For example, as shown, the straps 812 may lace in a crisscross pattern or X-shape across the tunnel 816 and the posterior portion of the cardioprotective apparel 800, running between layers of fabric forming the cardioprotective apparel 800.

In implementations, the tunnel 816 may be a tunnel stitched onto the posterior or back surface 818 of the cardioprotective apparel 800, as illustrated in FIG. 16A. In implementations, the tunnel 816 may be provided between layers of the cardioprotective apparel 800 (e.g., as illustrated in FIG. 16B). The tunnel 816 and/or the straps 812 may also include catches to prevent the straps 812 from being pulled through the tunnel 816. As an example, the ends of the straps 812 may be too wide to fit through the tunnel 816 or may include a piece (e.g., a plastic or metal piece) that is too large to fit through the tunnel 816.

The strap 812 may then attach to the cardioactive band 804 via the front adjustment mechanism 814 at least partially located on the cardioactive band 804. For example, the front adjustment mechanism 814 may include hook fabric located on an underside of the strap 812 (e.g., the non-viewable face of the strap 812 in FIG. 16A) and loop fabric located on the cardioactive band 804. As such, the patient 104 can adjust the fit of the cardioprotective apparel by removing the straps 812 from the cardioactive band 804 at the front adjustment mechanism 810, pulling the straps tighter or looser, and reattaching the straps 812 to the cardioactive belt at the front adjustment mechanism 810. This front adjustment mechanism (e.g., with hook-and-loop fabric) may be configured for case of placement and adjustment over the course of wear. In implementations, the patient 104 should only need adjust the cardioprotective apparel for water retention within a given size. The front straps 814 being adjustable at the front adjustment mechanisms 810 may allow the patient 104 to modify the sizing of the cardioprotective apparel 800 in one movement and to avoid interfering with and/or layering with a bra worn by the patient 104. Additionally, the front adjustment mechanisms 810 being provided on the front of the cardioactive band 804 may allow the patient 104 to more easily change the fit of the cardioprotective apparel 800 while the patient 104 is wearing the cardioprotective apparel 800 and may be particularly beneficial for patients with dexterity issues (e.g., caused by arthritis). By adjusting the straps 812, the patient 104 may also change length and/or shape of the respective shoulder harnesses 802 to provide for a better fit for the patient 104.

Moreover, in implementations, the shoulder harnesses 802 themselves may serve as the force applicators shifting the breast tissue of the patient away from predetermined anatomical regions (e.g., through the cut, pattern, construction, materials, elasticity, etc. of the shoulder harnesses 802). As such, adjusting the straps 812 using the adjustment mechanisms 810 to adjust the shape of the shoulder harnesses 802 may also adjust the ability the shoulder harnesses 802 to shift the breast tissue of the upper torso region of the patient. For example, tightening the straps 812 may provide an increased shift to the patient's breast tissue. In implementations, the cardioprotective apparel 800 may include a tissue sling similar to the sling portions 606 and 706 discussed above. In such implementations, adjusting the straps 812 may also adjust the shape of the tissue slings and/or the amount that the tissue slings shift the breast tissue of the patient 104.

FIG. 16B illustrates views of another implementation of the cardioprotective apparel 800, referred to in FIG. 16B as cardioprotective apparel 800a. As shown in FIG. 16B, the cardioprotective apparel 800a may be configured similarly to the cardioprotective apparel 800 but with a tunnel 816 that runs between layers of fabric forming the back surface 818 of the cardioprotective apparel 800a. Additionally, the lower portion of the back surface 818 includes a ventilated panel 821 configured to contact the lower back of the patient 104 when worn. The ventilated panel 821 may help the patient 104 remain cooler and thus more comfortable while wearing the cardioprotective apparel 800a.

FIGS. 16C-16F illustrate views of another implementation of the cardioprotective apparel 800, referred to in FIGS. 16C-16F as cardioprotective apparel 800b. The cardioprotective apparel 800b is generally configured similarly to the cardioprotective apparel 800, with straps 812 crisscrossing through the tunnel 816 on the back surface 818 of the cardioprotective apparel 800b. However, as shown in FIG. 16C, the front closure 806 may be conical or tapered like the front closure 622 shown in FIG. 13C and the front closure 708 shown in FIG. 15C. Additionally, the ends of the straps 812 may include pull tabs 823 configured to help the patient 104 detach the straps 812 from the cardioactive band 804.

In implementations, the electrode belt 300 is assembled into the cardioprotective apparel 800b using an inside surface of the cardioprotective apparel 800b (e.g., the surface configured to contact the patient's skin when the patient 104 wears the cardioprotective apparel 800b). The view in FIG. 16C illustrates receptacles 820a configured to receive therapy electrodes when the electrode belt 300 is assembled into the cardioprotective apparel 800b. In addition, FIGS. 16E and 16F illustrate the inner side of the cardioprotective apparel 800b that is used for assembly of the electrode belt 300 into the cardioprotective apparel 800b, where the inside surface includes a protective interior band flap 828. As shown in FIG. 16E (where the band flap 828 is closed) and 16F (where the band flap 828 is partially open), the interior band flap 828 can be attached and detached using fasteners 825. For example, FIG. 16F illustrates fasteners 825 including portions of hook fastener 825a provided on one side of the band flap 828 and strips of loop fastener 825b provided on the mating side of the cardioactive band 804. Additionally, as shown in FIG. 16F, in implementations the cardioprotective apparel 800b may include multiple band flaps 828, which may allow for easier assembly of the monitoring and treatment device 100 (e.g., by allowing the patient 104 to open and close smaller portions of band flaps 828 instead of one long band flap over the cardioactive band 804). Thus, for example, the cardioprotective apparel 800b may include a first band flap 828a configured to cover some of the sensing electrodes and the anterior therapy electrode and a second band flap 828b configured to cover the remaining sensing electrodes and the signal processing unit. In implementations, the first band flap 828a and the second band flap 828b may overlap to provide for a continuous covering over the cardioactive band 804.

When the band flap 828 is open, the electrode belt 300 can be assembled into the cardioprotective apparel 800b, for example, by inserting the therapy electrodes into the therapy electrode receptacles 822a (e.g., including inserting at least one anterior therapy electrode into an anterior therapy electrode receptacle on the cardioactive band 804 and inserting at least one posterior therapy electrode into a posterior therapy electrode receptacle on the back surface 818 of the cardioprotective apparel 800b) and attaching sensing electrodes to sensing electrode receptacles 822b. The band flap 828 can then be closed over the assembled electrode belt 300 to hold the components of the electrode belt in place and keep non-electrode components off of the patient's skin, thereby making the monitoring and treatment device 100 more comfortable to wear. In this sense, the receptacles 822 may be configured differently from the receptacles 614 and 718 discussed above in that the receptacles 822 are configured to be accessible from the inner side of the cardioprotective apparel 800b (via the band flap 828). Increased patient comfort may lead to longer term patient compliance with wearing and using the monitoring and treatment device 100 over the prescribed period of wear. To ensure that the electrodes maintain contact with the patient's skin, the band flap 828 also includes sensing electrode openings 824 that fit over the sensing electrodes to keep the sensing electrodes in place and allow the sensing electrodes to contact the patient's skin through the openings 824. Similarly, the band flap 828 includes a therapy electrode opening 826 through which the anterior therapy electrode can contact the patient's skin. Similar to the features discussed above with respect to the cardioprotective apparel 600 (as well as the cardioprotective apparel 600a and 600b) and the cardioprotective apparel 700 (as well as the cardioprotective apparel 700a and 700b), these features of the cardioprotective apparel 800, 800a, and 800b may help the components of the electrode belt 300 remain in place against desired anatomical locations and resist displacing forces, such as transverse rotational forces exerted by underlying tissue of the patient's upper torso region.

In implementations, to ensure a better fit of the cardioprotective apparel and a desired compression of the cardioprotective apparel to keep the electrodes in good skin contact, the cardioprotective apparel may a number of material zones, where each zone has a predetermined elasticity. In implementations, the material zones include one or more zones corresponding to a portion of patient anatomy. Each of these patient anatomy zones may have a corresponding elasticity configured to accommodate the corresponding portion of patient anatomy. For example, at least one zone may include the force applicator configured to shift the breast tissue of the upper torso region of the patient. In implementations, the material zones may additionally or alternatively include one or more zones corresponding to a component of the electrode belt 300, where each of these electrode belt zone has a corresponding elasticity configured to provide a desired and predetermined compression over the associated electrode belt component. For example, electrode belt zones may include zones configured to provide a predetermined compression to at least one sensing electrode and/or at least one therapy electrode.

In implementations, the elasticity of each zone is at least partially controlled by the material forming each zone. In implementations, each material zone may be formed of a different material. In implementations, at least some of the zones may be formed of the same material but with the material structured to provide a different elasticity for the zone. For example, each zone may formed of a different material knit configured to provide a desired elasticity to the corresponding zone. Other options include using different material thicknesses, other types of weaves, layers, and/or the like.

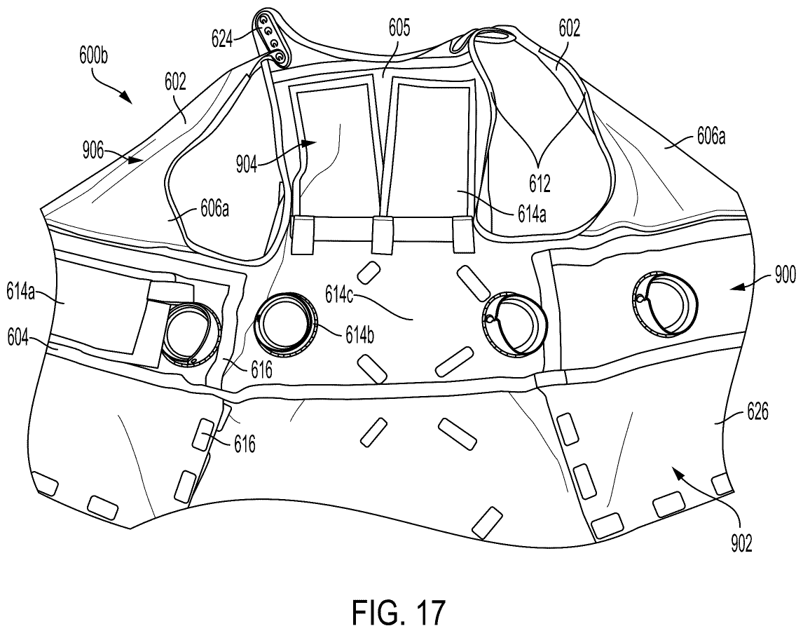

As an illustration of material zones, FIG. 17 shows another view of the cardioprotective apparel 600b. Specifically, the view in FIG. 17 is of the cardioprotective apparel 600b as shown of the exterior side of the back surface 605 with the exterior flap 626 opened (e.g., configured as a full-length back panel, similar to the back panel 716) to reveal receptacles 614. In the example shown in FIG. 17, the receptacles 614 include therapy electrode receptacles 614a, sensing electrode receptacles 614b (e.g., configured such that sensing electrodes can be popped, snapped, twisted, etc. into the sensing electrode receptacles 614b), and a signal processing unit receptacle 614c (e.g., configured as a pocket that a signal processing unit can sit in when the exterior flap 626 is closed). The cardioprotective apparel 600b also includes different material zones corresponding to portions of patient anatomy and/or corresponding to certain electrode belt components. For instance, a first zone 900 includes the main body of the cardioprotective apparel 600b. A second zone 902 includes the exterior flap 626. A third zone 904 corresponds to the therapy electrode receptacles 614a. A fourth zone corresponds to the force applicators 606a.

Each of the material zones 900, 902, 904, and 906 may be formed of a different material. As an example, the first zone 900 may be formed of a material configured for lightweight moisture management (e.g., a 195G Turbo Dry Mesh). The second zone 902 may be formed of a compression knit to hold the electrode belt components in place and against the patient's skin (e.g., a 240G jersey with Fiber J spandex). The third zone 904 may be formed of a compression knit to hold the therapy electrodes against conductive mesh on the side of the therapy electrode receptacle 614a configured to contact the patient's skin, with the conductive mesh shown in FIG. 13C (e.g., a 140G knit). The fourth zone 906 may be formed of a material configured for moisture management with gentle compression to direct breast tissue forward and away from sensing and therapy electrode anatomical regions on the body (e.g., a double-layered 117G wicking fabric).

In implementations, other material zones and/or other materials may be used. For example, with respect to the cardioprotective apparel 700, the cardioactive band 704 and the therapy electrode receptacles 718a may be formed of a material configured to provide even compression around the body with breathability (e.g., a 280G double knit). The back panel 716 and the shoulder harnesses 702 may be formed of a material configured for breathability and, for the shoulder harnesses 702, cushioning (e.g., a 300G honeycomb spacer fabric). The slings portions 706 may be formed of a material configured for next-to-skin breathability and comfort, for example, when worn with a bra (e.g., a 135G mini-check mesh). In examples, the cardioprotective apparel 700 may include additional material zones, such as varying compression zones around the arm holes, main body of the cardioprotective apparel 700, and portion of the cardioprotective apparel 700 configured to contact the patient's chest, which can be formed using an engineered knit full garment solution.

As another example, with respect to the cardioprotective apparel 800, the main body of the cardioprotective apparel may be formed of a material configured for an even compression around the body with breathability (e.g., a 250G knit). The interior band flap 828 may be formed of a high-spandex compression knit (e.g., a 280G double knit). In examples, the cardioprotective apparel 800 may similarly include additional material zones like zones around the arm holes, main body, and chest formed using an engineered full garment solution.

In implementations, cardioprotective apparel can include additional zones to the material zones described above, such as zones configured to accommodate front torso protections. As another example, in implementations, the cardioactive band of the cardioprotective apparel may form one zone, as described above. However, in other implementations, the cardioactive band of the cardioprotective apparel may include two or more zones. For instance, an anterior portion of the cardioactive band may be a first zone with a first, higher elasticity configured to accommodate front torso projections for larger sizes. A posterior portion of the cardioactive band may be a second zone with a second, lower elasticity configured to provide compression on the anterior part of the patient's torso. As another example, a zone may include at least a portion of the shoulder harnesses of the cardioprotective apparel, such as at least a portion of the at least two shoulder harnesses that is configured to shift the breast tissue of the upper torso region of the patient 104. These shoulder harness zones may include tissue slings and/or other features of the shoulder harnesses configured to shift the patient's breast tissue, such as materials forming portions of a shoulder harness configured as a force applicator.

In implementations, features from various examples of cardioprotective apparel may be combined, subtracted, and/or swapped out. Such modifications to the cardioprotective apparel may allow, for example, for better accommodation of patient anatomy, for better shaping and/or shifting of patient anatomy away from predetermined anatomical regions for electrodes or other components of the monitoring and treatment device 100, to allow the monitoring and treatment device 100 to be more comfortably and effectively worn with a bra, and/or the like. Other features may also or alternatively be added to the cardioprotective apparel.

In implementations, the cardioprotective apparel may also be configured in a range of sizes. The patient 104 may be fitted for cardioprotective apparel when the patient 104 receives the monitoring and treatment device 100, with a technician choosing a size of cardioprotective apparel for the patient 104 based on their fitting. In implementations, the cardioprotective apparel may also be configured to shift the patient's breast tissue according to the size of the cardioprotective apparel. For example, larger sizes may include a sling portion with a different elasticity to allow for more breast tissue displacement, assuming that patients in larger sizes may have more breast tissue compared to smaller sizes. In implementations, the length of an anterior portion of the cardioprotective apparel's cardioactive band may range between about 8.5 inches and 19 inches between sizes. The length of a posterior portion of the cardioactive band may range between about 17 inches and 38 inches between sizes.

Additionally, in implementations, the cut and pattern of the cardioprotective apparel may be configured to accommodate different types of patient anatomy common across patient sizes. For example, for larger sizes, the cardioactive band may be contoured to accommodate front torso projections. Further, the cut and pattern of the cardioprotective apparel may be configured for the ways that male patient anatomy changes across patient sizes and female patient anatomy changes across patient sizes. To illustrate, the cardioprotective apparel may be provided in unisex small sizes. However, for medium and larger sizes, the cardioprotective apparel may be provided in male sizes and female sizes patterned to accommodate the way the male upper torso region changes in medium and larger sizes versus the way the female upper torso region changes in medium and larger sizes. As an example, male anatomy may tend to grow more uniformly around the torso in larger sizes and may include front torso projections formed of harder tissue. Accordingly, male sizes may include cardioactive bands contoured to accommodate male front torso projections, both in terms of shape and tissue composition. By contrast, female anatomy may tend to grow more in the anterior portion of the upper torso than in the posterior portion of the upper torso. Moreover, female front torso projection may be formed of softer tissues. As such, female sizes may include cardioactive bands contoured to accommodate female front torso projections, both in terms of shape and tissue composition.

Returning to the cardiac device controller 106 and the controller 400, FIG. 18 illustrates a sample component-level view of the cardiac device controller 1000. In implementations, the cardiac device controller 106 and/or the controller 400 may be configured similarly to the cardiac device controller illustrated in FIG. 18. As shown in FIG. 18, the cardiac device controller 1000 may include a housing 1002 configured to house a number of electronic components, including a sensor interface 1004, a data storage 1006, a network interface 1008, a user interface 1010, at least one battery 1012 (e.g., positioned within a battery chamber configured for such a purpose), a cardiac event detector 1014, an alarm manager 1016, at least one processor 1018, and a therapy delivery circuit 1020. In some implementations, rather than being a monitoring and treatment device 100, the patient 104 may be provided with a similar ambulatory monitoring device that includes like components to those described above but may not include therapeutic components. That is, in some implementations, an ambulatory monitoring device can include ECG monitoring components and not provide therapy to the patient 100. Accordingly, the ambulatory monitoring device may not include the therapy delivery circuit 1020 and the therapy electrodes 114 (shown in dotted lines), or the therapy delivery circuit 1020 and the therapy electrodes 114 may be disconnectable from the rest of the ambulatory device. However, the ambulatory monitoring device may include other monitoring components as described here with respect to FIG. 18 and with respect to the figures above.

In implementations, the processor 1018 includes one or more processors (or one or more processor cores) that are each configured to perform a series of instructions that result in the manipulation of data and/or the control of the operation of the other components of the cardiac device controller 1000. In implementations, when executing a specific process (e.g., monitoring sensed biosignals), the processor 1018 can be configured to make specific logic-based determinations based on input data received. The processor 1018 may be further configured to provide one or more outputs that can be used to control or otherwise inform subsequent processing to be carried out by the processor 1018 and/or other processors or circuitry to which the processor 1018 is communicably coupled. Thus, the processor 1018 reacts to a specific input stimulus in a specific way and generates a corresponding output based on that input stimulus. In example cases, the processor 1018 can proceed through a sequence of logical transitions in which various internal register states and/or other bit cell states internal or external to the processor 1018 may be set to logic high or logic low.

As referred to herein, the processor 1018 can be configured to execute a function where software is stored in a data store (e.g., the data storage 1006) coupled to the processor 518, the software being configured to cause the processor 1018 to proceed through a sequence of various logic decisions that result in the function being executed. The various components that are described herein as being executable by the processor 1018 can be implemented in various forms of specialized hardware, software, or a combination thereof. For example, the processor 1018 can be a digital signal processor (DSP) such as a 24-bit DSP processor. As another example, the processor 1018 can be a multi-core processor, e.g., having two or more processing cores. As another example, the processor 1018 can be an Advanced RISC Machine (ARM) processor, such as a 32-bit ARM processor. The processor 1018 can execute an embedded operating system and further execute services provided by the operating system, where these services can be used for file system manipulation, display and audio generation, basic networking, firewalling, data encryption, communications, and/or the like.

The data storage 1006 can include one or more of non-transitory media, such as flash memory, solid state memory, magnetic memory, optical memory, cache memory, combinations thereof, and others. The data storage 1006 can be configured to store executable instructions and data used for operation of the cardiac device controller 1000. In implementations, the data storage 1006 can include sequences of executable instructions that, when executed, are configured to cause the processor 1018 to perform one or more functions. Additionally, the data storage 1006 can be configured to store information such as sensed biosignals and biosignal-based data, as well as data from other sensors of the monitoring and treatment device 100, as described in further detail below.

In examples, the network interface 1008 can facilitate the communication of information between the cardiac device controller 1000 and one or more devices or entities over a communications network. For example, the network interface 1008 can be configured to communicate with a remote server or other similar computing device. As using, using the network interface 1008, the monitoring and treatment device 100 may transmit data, such as sensed ECG signals and/or treatment determinations, to the remote server. In implementations, the network interface 1008 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the monitoring and treatment device 100). The intermediary device(s) may in turn communicate the data to the remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server over a Wi-Fi communications link based on the IEEE 802.11 standard. In implementations, the network interface 1008 may be configured to instead communicate directly with the remote server without the use of intermediary device(s). In such implementations, the network interface 1008 may use any of the communications links and/or protocols provided above to communicate directly with the remote server.

The sensor interface 1004 can include physiological signal circuitry that is coupled to one or more externally applied sensors 1022. The externally applied sensors 1022 may include, for example, one or more externally sensors configured to sense one or more biosignals from the patient 104. As shown, the sensors may be coupled to the cardiac device controller 1000 via a wired or wireless connection. The externally applied sensors 1022 may include one or more ECG electrodes 102 configured to sense cardiac activity of the patient 100; one or more cardiovibration sensors 1026 configured to sense cardiovibrations of the patient 100 such as heart sounds caused by the opening and/or closing of valves in the patient's heart; one or more tissue fluid monitors 1028 configured to use, for example, radiofrequency (RF) waves to detect a tissue fluid level in the patient's thoracic cavity based on attenuation of the RF waves; and/or the like. Other examples of externally applied sensors 520 may include a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

For example, in implementations and as described briefly above, the one or more cardiovibration sensors 1026 can be configured to detect cardiac or pulmonary vibration information. The one or more cardiovibration sensors 1026 can transmit information descriptive of the cardiovibrations (and other types of sensed vibrations) to the sensor interface 1004 for subsequent analysis. For example, the one or more cardiovibration sensors 1026 can detect the patient's heart valve vibration information (e.g., from opening and closing during cardiac cycles). As a further example, the one or more cardiovibration sensors 1026 can be configured to detect cardiovibrational signal values including one or more of S1, S2, S3, and S4 cardiovibrational biomarkers. From these cardiovibrational signal values or heart vibration values, certain heart vibration metrics may be calculated (e.g., at the monitoring and treatment device 100 and/or at a remote server in communication with the monitoring and treatment device 100). These heart vibration metrics may include one or more of electromechanical activation time (EMAT), average EMAT, percentage of EMAT (% EMAT), systolic dysfunction index (SDI), or left ventricular systolic time (LVST). The one or more cardiovibration sensors 1026 can also be configured to detect heart wall motion, for instance, by placement of the sensor in the region of the apical beat. In implementations, the one or more cardiovibration sensors 1026 can include vibrational sensor configured to detect vibrations from the patient's cardiac and pulmonary system and provide an output signal responsive to the detected vibrations of a targeted organ. For example, the one or more cardiovibration sensors 1026 may be configured to detect vibrations generated in the trachea or lungs due to the flow of air during breathing. In implementations, additional physiological information can be determined from pulmonary-vibrational signals such as, for example, lung vibration characteristics based on sounds produced within the lungs (e.g., stridor, crackle, etc.). In implementations, the one or more cardiovibration sensors 1026 can include a multi-channel accelerometer, for example, a three-channel accelerometer configured to sense movement in each of three orthogonal axes such that patient movement/body position can be detected and correlated to detected cardiovibrations information.

In implementations, the sensor interface 1004 may be connected to one or more motion sensors (e.g., one or more accelerometers, gyroscopes, magnetometers, ballistocardiographs, etc.) as part of the externally applied sensors 1022. In implementations, the cardiac device controller 1000 may include a motion detector interface, either implemented separately or as part of the sensor interface 504. For instance, as shown in FIG. 8, the cardiac device controller 1000 may include a motion sensor interface 1024 operatively coupled to one or more motion sensors 1030 configured to generate motion data, for example, indicative of physical activity performed by the patient 104 and/or physiological information internal to the patient 104. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As an illustration, the motion data may include accelerometer counts indicative of physical activity performed by the patient 104, accelerometer counts indicative of respiration rate of the patient 104, accelerometer counts indicative of posture information for the patient 104, accelerometer counts indicative of cardiovibrational information for the patient 104, and/or the like.

The motion sensor interface 1024 is configured to receive one or more outputs from the motion sensors 1030. The motion sensor interface 1024 can be further configured to condition the output signals by, for example, converting analog signals to digital signals (if using an analog motion sensor), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In examples, the motion sensor interface 1024 can be configured to filter the signals using a high-pass or band-pass filter to isolate the acceleration of the patient due to movement from the component of the acceleration due to gravity. Additionally, the motion sensor interface 1024 can configure the outputs from the motion sensor(s) 1030 for further processing. For example, the motion sensor interface 1024 can be configured to arrange the output of an individual motion sensor 1030 as a vector expressing acceleration components of the x-axis, the y-axis, and the z-axis of the motion sensor 1030. The motion sensor interface 1024 can thus be operably coupled to the processor 1018 and configured to transfer the output and/or processed motion signals from the motion sensors 1030 to the processor 1018 for further processing and analysis.

In implementations, the one or more motion sensors 1030 can be integrated into one or more components of the monitoring and treatment device 101, either within the cardiac device controller 1000 or external to the cardiac device controller 1000 as shown in FIG. 18. For instance, in some implementations, the one or more motion detectors 1030 may be located in or near the ECG electrodes 102. In some implementations, the one or more motion detectors 1030 may be located elsewhere on the monitoring and treatment device 100. For example, a motion detector 1030 may be integrated into the cardiac device controller 1000 (e.g., such that the one or more motion detectors 1030 would be located within the housing 1002 of the cardiac device controller 1000, as shown in FIG. 18). In some implementations, a motion detector 1030 may be integrated into another component of the monitoring and treatment device 100, such as a therapy electrode 114, the signal processing unit 108, and/or the like.

As described above, the sensor interface 1004 and the motion sensor interface 1024 can be coupled to any one or combination of external sensors to receive patient data indicative of patient parameters. Once data from the sensors has been received by the sensor interface 1004 and/or the motion sensor interface 1024, the data can be directed by the processor 1018 to an appropriate component within the cardiac device controller 1000. For example, ECG signals collected by the ECG sensors 102 may arrive at the sensor interface 1004, and the sensor interface 1004 may transmit the ECG signals to the processor 1018, which, in turn, relays the patient's ECG data to the cardiac event detector 1014 (e.g., described in further detail below). The sensor data can also be stored in the data storage 1006 and/or transmitted to a remote server via the network interface 1008.

In implementations, the user interface 510 as shown in FIG. 18 may include one or more physical interface devices, such as input devices, output devices, and combination input/output devices, and a software stack configured to drive operation of the devices. These user interface elements may render visual, audio, and/or tactile content. Thus, the user interface 1010 may receive inputs and/or provide outputs, thereby enabling a user to interact with the medical device controller 1000. For example, as described in further detail below, the user interface 1010 may include one or more response buttons that the patient 104 can press in response to an alarm to indicate that the patient 104 is still conscious.

The cardiac device controller 1000 can also include at least one battery 1012 configured to provide power to one or more components integrated in the cardiac device controller 1000. The battery 1012 can include a rechargeable multi-cell battery pack. In one example implementation, the battery 1012 can include three or more cells (e.g., 2200 mA lithium ion cells) that provide electrical power to the other device components within the cardiac device controller 1000. For example, the battery 1012 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the cardiac device controller 1000.

The alarm manager 1016 can be implemented using hardware or a combination of hardware and software. For instance, in some examples, the alarm manager 1016 can be implemented as a software component that is stored within the data storage 1006 and executed by the processor 1018. In this example, the instructions included in the alarm manager 1016 can cause the processor 1018 to configure alarm profiles and notify intended recipients using the alarm profiles. In other examples, the alarm manager 1016 can be an application-specific integrated circuit (ASIC) that is coupled to the processor 1018 and configured to manage alarm profiles and notify intended recipients using alarms specified within the alarm profiles. Thus, examples of the alarm manager 1016 are not limited to a particular hardware or software implementation.

The therapy delivery circuit 1020 can be coupled to the therapy electrodes 114 and configured to provide therapy to the patient 104. For example, the therapy delivery circuit 1020 can include, or be operably connected to, circuitry components that are configured to generate and provide an electrical therapeutic shock. The circuitry components can include, for example, resistors, capacitors, relays and/or switches, electrical bridges such as an H-bridge (e.g., including a plurality of insulated gate bipolar transistors or IGBTs), voltage and/or current measuring components, and other similar circuitry components arranged and connected such that the circuitry components work in concert with the therapy delivery circuit 1020 and under the control of one or more processors (e.g., processor 1018) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

In implementations, the therapy delivery circuit 1020 includes a first high-voltage circuit connecting a first pair of the therapy electrodes 114 and a second high-voltage circuit connecting a second pair of the therapy electrodes 114 such that the first biphasic therapeutic pulse is delivered via the first high-voltage circuit and the second biphasic therapeutic pulse is delivered via the second high-voltage circuit. In implementations, the second high-voltage circuit is configured to be electrically isolated from the first high-voltage circuit. In implementations, the therapy delivery circuit 1020 includes a capacitor configured to be selectively connected to the first high-voltage circuit and/or the second high-voltage circuit. As such, the first high-voltage circuit may powered by the capacitor when the capacitor is selectively connected to the first high-voltage circuit, and the second high-voltage circuit may be powered by the capacitor when the capacitor is selectively connected to the second high-voltage circuit. In implementations, the therapy delivery circuit 1020 includes a first capacitor electrically connected to the first high-voltage circuit and a second capacitor electrically connected to the second high-voltage circuit.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 μF or larger, or four capacitors of approximately 650 μF can be used. The capacitors can have a 1600 VDC or higher rating for a single capacitor, or a surge rating between approximately 350 to 500 VDC for paralleled capacitors and can be charged in approximately 15 to 30 seconds from a battery pack.

In implementations, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 1020 can be configured to perform the switching and pulse delivery operations, e.g., under control of the processor 1018. As the energy is delivered to the patient 104, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 1020 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

In implementations, the cardiac event detector 1014 can be configured to monitor the patient's ECG signal for an occurrence of a cardiac event such as an arrhythmia or other similar cardiac event. The cardiac event detector 1014 can be configured to operate in concert with the processor 1018 to execute one or more methods that process received ECG signals from, for example, the ECG sensing electrodes 102 and determine the likelihood that the patient 104 is experiencing a cardiac event, such as a treatable arrhythmia. The cardiac event detector 1014 can be implemented using hardware or a combination of hardware and software. For instance, in some examples, the cardiac event detector 1014 can be implemented as a software component that is stored within the data storage 1006 and executed by the processor 1018. In this example, the instructions included in the cardiac event detector 1014 can cause the processor 1018 to perform one or more methods for analyzing a received ECG signal to determine whether an adverse cardiac event is occurring, such as a treatable arrhythmia. In other examples, the cardiac event detector 1014 can be an application-specific integrated circuit (ASIC) that is coupled to the processor 1018 and configured to monitor ECG signals for adverse cardiac event occurrences. Thus, examples of the cardiac event detector 1014 are not limited to a particular hardware or software implementation.

In response to the cardiac event detector 1014 determining that the patient 104 is experiencing a treatable arrhythmia, the processor 1018 is configured to deliver a cardioversion/defibrillation shock to the patient 104 via the therapy electrodes 114. In implementations, the alarm manager 1016 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., one or more remote servers, such as monitoring systems or emergency response systems). For example, when the processor 1018 determines using data from the ECG sensing electrodes 102 that the patient 104 is experiencing a treatable arrhythmia, the alarm manager 1016 may issue an alarm via the user interface 1010 that the patient 104 is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient 104 that the patient 104 can abort the delivery of the defibrillating shock by interacting with the user interface 1010. For instance, the patient 104 may be able to press a user response button or user response buttons on the user interface 1010, after which the alarm manager 1016 will cease issuing an alert and the processor 1018 will no longer prepare to deliver the defibrillating shock.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-130. (canceled)

131. An ambulatory patient monitoring and treatment device configured to be worn continuously by a patient, comprising:
a plurality of ECG sensing electrodes configured to sense cardiac activity of the patient;
a plurality of therapy electrodes configured to deliver one or more electrical therapeutic shocks to the patient;
cardioprotective apparel configured to be donned about an upper torso region of the patient, wherein the cardioprotective apparel comprises
a cardioactive band configured to be disposed on an inferior end of the upper torso region of the patient and support
at least one anterior therapy electrode of the plurality of therapy electrodes such that the at least one anterior therapy electrode is disposed on an anterior therapy electrode placement region when the device is worn by the patient, and
the plurality of ECG sensing electrodes distributed in a spaced apart configuration such that the plurality of ECG sensing electrodes are disposed on predetermined ECG sensing anatomical regions when the device is worn by the patient, and
at least two shoulder harnesses attached to the cardioactive band and configured to support the cardioprotective apparel on the patient's shoulders,
wherein each of the at least two shoulder harnesses comprises a force applicator having an elasticity configured to shift breast tissue of the upper torso region of the patient away from at least one of
the anterior therapy electrode placement region on the patient, or
at least one of the predetermined ECG sensing anatomical regions on the patient, and
wherein the shifting of the breast tissue of the upper torso region of the patient is configured to facilitate the at least one anterior therapy electrode and the plurality of ECG sensing electrodes to lie in a flush manner with the patient's skin when the device is worn by the patient, and
a device controller and associated circuitry operably connected to the plurality of ECG sensing electrodes and the plurality of therapy electrodes and configured to determine whether the patient is experiencing a treatable arrhythmia based on the sensed cardiac activity and instruct the delivery of the one or more electrical therapeutic shocks to the patient via the plurality of therapy electrodes in response to determining that the patient is experiencing a treatable arrhythmia.

132. The device of claim 131, wherein each force applicator comprises a sling portion configured to shift the breast tissue of the upper torso region of the patient.

133. The device of claim 132, wherein the sling portion comprises a fabric having the elasticity configured to shift the breast tissue of the upper torso region of the patient.

134. The device of claim 133, wherein the fabric of the sling portion comprises at least one of a mesh, a knit, or a stretch woven.

135. The device of claim 131, wherein each force applicator comprises at least one of a cut, a pattern, a construction, a material, or a material elasticity of the corresponding shoulder harness.

136. The device of claim 131, wherein each force applicator is configured to provide less than 0.65 psi of pressure to the breast tissue of the upper torso region of the patient.

137. The device of claim 131, wherein the at least two shoulder harnesses are configured to minimize interference with a bra worn by the patient concurrently with the monitoring and treatment device.

138. The device of claim 137, wherein the at least two shoulder harnesses are configured to be disposed along lateral ends of the upper torso region of the patient when the device is worn by the patient to minimize overlap between the bra worn by the patient and the at least two shoulder harnesses.

139. The device of claim 137, wherein the at least two shoulder harnesses are configured to stabilize the breast tissue within the bra when the bra is worn over the monitoring and treatment device.

140. The device of claim 131, wherein the cardioprotective apparel comprises a plurality of zones; and
wherein each zone comprises a predetermined elasticity for the respective zone.

141. The device of claim 140, wherein the plurality of zones comprises a zone corresponding to a portion of patient anatomy; and
wherein the predetermined elasticity of the zone corresponding to the portion of patient anatomy is configured to accommodate the corresponding portion of patient anatomy.

142. The device of claim 140, wherein the plurality of zones comprises a zone corresponding to one or more of at least one ECG sensing electrode or at least one therapy electrode; and
wherein the predetermined elasticity of the zone corresponding to the one or more of the at least one sensing electrode or the at least one therapy electrode is configured to provide a predetermined compression to the one or more of the at least one sensing electrode or the at least one therapy electrode.

143. The device of claim 140, wherein the plurality of zones comprises a first zone comprising each of the force applicators configured to shift the breast tissue of the upper torso region of the patient.

144. The device of claim 131, wherein the cardioactive band further comprises at least one anterior therapy electrode receptacle configured to receive a respective anterior therapy electrode and resist a transverse rotational force exerted by underlying tissue of the upper torso region of the patient such that the respective anterior therapy electrode is configured to lie in the flush manner with the patient's skin when the device is worn by the patient.

145. The device of claim 144, wherein the at least one anterior therapy electrode receptacle comprises at least one anterior pocket disposed on an anterior portion of the cardioactive band.

146. The device of claim 145, wherein the cardioprotective apparel comprises an inner side configured to face the patient's skin and an outer side configured to face away from the patient's skin; and
wherein the at least one anterior pocket disposed on the anterior portion of the cardioactive band is configured to be accessible from the outer side of the cardioprotective apparel.

147. The device of claim 145, wherein the cardioprotective apparel comprises an inner side configured to face the patient's skin and an outer side configured to face away from the patient's skin; and
wherein the at least one anterior pocket disposed on the anterior portion of the cardioactive band is configured to be accessible from the inner side of the cardioprotective apparel.

148. The device of claim 145, wherein each anterior pocket is pre-gathered, the pre-gathering facilitating the respective anterior therapy electrode in lying in the flush manner with the patient's skin when the respective anterior therapy electrode is received into the anterior pocket and the device is worn by the patient.

149. The device of claim 131, wherein the cardioactive band is configured to be contoured to the upper torso region of the patient.

150. The device of claim 149, wherein the cardioactive band is configured to be contoured to accommodate a female front torso projection.
